(19) United States
(12) Patent Application Publication
(10) Pub. No.: US 2026/0364125 A1
(43) Pub. Date: Nov. 19, 2026

(54) MICROBIAL COMMUNITIES THAT INHIBIT CLOSTRIDIOIDES DIFFICILE AND METHODS OF USING SAME

(71) Applicant: Wisconsin Alumni Research Foundation, Madison, WI (US)

(72) Inventors: Ophelia Venturelli, Chapel Hill, NC (US); Yiyi Liu, Cranberry, NJ (US); Jaron Thompson, Madison, WI (US); Gabriel Miranda, Santiago (CL)

(73) Assignee: Wisconsin Alumni Research Foundation, Madison, WI (US)

(21) Appl. No.: 19/670,856

(22) Filed: May 7, 2026

Related U.S. Application Data
(60) Provisional application No. 63/801,393, filed on May 7, 2025.

(51) Int. Cl. A61K 35/74; A61K 35/00; A61P 31/04; C12N 1/20; C12R 1/19

(52) U.S. Cl. CPC A61K 35/74; A61P 31/04; C12N 1/20; A61K 2035/11; C12R 2001/19

(57) ABSTRACT

Microbial communities that inhibit Clostridioides difficile and methods of using the microbial communities to inhibit C. difficile.

### CROSS-REFERENCE TO RELATED APPLICATIONS

Priority is hereby claimed to U.S. Provisional Application 63/801,393, filed May 7, 2025, which is incorporated herein by reference in its entirety.

### STATEMENT REGARDING FEDERALLY SPONSORED RESEARCH

This invention was made with government support under AI156438, AI159980, and GM124774 awarded by the National Institutes of Health. The government has certain rights in the invention.

### FIELD OF THE INVENTION

The invention is directed to microbial communities that inhibit Clostridioides difficile and methods of using the microbial communities to inhibit C. difficile.

### BACKGROUND

Clostridioides difficile is an opportunistic pathogen that causes diarrhea and colitis (inflammation of the colon) or C. difficile infection (CDI). Most cases of CDI occur during or shortly after taking antibiotics. Together, antibiotics and C. difficile substantially disrupt the normal flora (microbiome) of the gut, which normally provides colonization resistance. As a result, there is a high recurrence rate of CDI in antibiotic-treated patients, thereby limiting the options for follow-on treatment.

Options for treating CDI include monoclonal antibodies that target a specific toxin produced by C. difficile. While such treatments can be effective, they are not without their limitations, so researchers continue to pursue alternatives.

One promising alternative is fecal microbiota transplantation (FMT), whereby gut microbiota from healthy donors is transplanted into the gastrointestinal tract of a patient. This approach has the effect of reestablishing the microbiome of the patient. However, variations in microbiome compositions across FMT donors leads to uncertainty in efficacy and safety (e.g., adverse health effects including death and transfer of antibiotic resistant bacteria).

Strategies for treating CDI that avoid the aforementioned problems are needed.

### SUMMARY OF THE INVENTION

One aspect of the invention is directed to a composition comprising a bacterial community.

In some embodiments, the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of: Escherichia coli (EC); and any two or more or any three or more of Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of: Escherichia coli (EC); Bacteroides uniformis (BU); and any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of: Escherichia coli (EC); Bacteroides uniformis (BU); and any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of: Escherichia coli (EC); and any one or more, any two or more, or each of Bacteroides uniformis (BU), Bacteroides fragilis (BF), and Roseburia intestinalis (RI).

In some embodiments, the bacterial community consists of individual bacterial cells of the purified bacterial strains and a number of the individual bacterial cells in the bacterial community is at least 50% of a total number of individual bacterial cells in the composition.

In some embodiments, no more than 5% of a total number of individual bacterial cells in the composition are bacterial strains of Acidaminococcus fermentans, Acidaminococcus intestine, Anaerostipes hadrus, Anaerotruncus colihominis DSM 17241, Anaerotruncus colihominis, Bifidobacterium bifidum, Blautia coccoides, Blautia faecis, Blautia hansenii, Blautia luti, Blautia producta ATCC 27340, Blautia producta, Blautia wexlerae, Clostridium bacterium UC5.1-1D4, Clostridium bolteae 90A9, Clostridium bolteae, Clostridium disporicum, Clostridium hathewayi, Clostridium hiranonis, Clostridium innocuum, Clostridium orbiscindens 1_3_50AFAA, Clostridium symbiosum WAL-14163, Clostridium symbiosum, Dracourtella massiliensis GDI, Dracourtella massiliensis, Eisenbergiella tayi, Erysipelatoclostridium ramosum, Erysipelotrichaceae_bacterium_21_3, Eubacterium contortum, Eubacterium fissicatena, Eubacterium rectale, Flavinofractor plautii, Fusicatenibacter saccharivorans, Lachnospiraceae bacterium 7-_I_58FAA, Lactobacillus mucosae, Marvinbryantaformexigens, Megasphaera elsdenii, Pseudoflavinofractor capillosus, Roseburia faecis, Ruminococcus albus, Ruminococcus champanellensis, Ruminococcus faecis, Ruminococcus obeum, Ruminococcus torques, Sellimonas intestinalis, Subdoligranulum, and Turicibacter sanguinis.

In some embodiments, no more than 5% of a total number of individual bacterial cells in the composition are bacterial strains of Parabacteroides johnsonii (PJ), Phocaeicola vulgatus (PV), Bacteroides cellulosilyticus (BY), Enterocloster asparagiformis (CG), Anaerobutyricum hallii (EH), Anaerostipes caccae (AC), Agathobacter rectalis (ER), Peptacetobacter hiranonis (CH), Eggerthella lenta (EL), Bifidobacterium pseudocatenulatum (BP), Bifidobacterium longum (BL), and Bifidobacterium adolescentis (BA).

In some embodiments, no more than 5% of a total number of individual bacterial cells in the composition are bacterial strains of Bacteroides thetaiotaomicron (BT), Bacteroides ovatus (BO), Bacteroides caccae (BC), Collinsella aerofaciens (CA), Coprococcus comes (CC), Faecalibacterium prausnitzii (FP), Segatella copri (PC), Dorea formicigenerans (DF), Blautia hydrogenotrophica (BH), Clostridium scindens (CS), Dorea longicatena (DL), and Desulfovibrio piger (DP).

In some embodiments, no more than 5% of a total number of individual bacterial cells in the composition are bacterial strains of Clostridium hiranonis, Bacteroides thetaiotaomicron, Bacteroides uniformis, Phocaeicola vulgatus, Clostridium scindens, Collinsella aerofaciens, and Desulfovibrio piger.

In some embodiments, each purified bacterial strain in the bacterial community is a commensal strain. In some embodiments, each bacterium in the composition is a commensal strain.

In some embodiments, one or more of the purified bacterial strains are in spore form. In some embodiments, each of the purified bacterial strains are in spore form. In some embodiments, one or more of the purified bacterial strains are in vegetative form. In some embodiments, each of the purified bacterial strains are in vegetative form.

In some embodiments, each of the purified bacterial strains is lyophilized.

In some embodiments, the bacterial community comprises bacterial strains purified from different sources.

In some embodiments, the bacterial community inhibits C. difficile toxin production. In some embodiments, the bacterial community inhibits C. difficile replication and/or survival.

In some embodiments, the composition is in the form of a pharmaceutical composition comprising the bacterial community in combination with a pharmaceutically acceptable excipient. In some embodiments, the composition is formulated for oral or rectal delivery. In some embodiments, the composition is formulated for delivery to the intestine or the colon. In some embodiments, the composition is in the form of a food product comprising the bacterial community and a nutrient.

Another aspect of the invention is directed to methods of treating a C. difficile infection in a subject.

Some embodiments comprise administering to the subject a therapeutically effective amount of the composition of any prior claim to treat the C. difficile infection.

In some embodiments, the subject is an asymptomatic C. difficile carrier.

In some embodiments, the subject is administered one or more doses of an antibiotic prior to administration of the composition. In some embodiments, the subject has not been administered an antibiotic prior to administration of the composition.

In some embodiments, the composition is administered to the subject by oral administration or rectal administration.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Iterative modeling framework to explore design space of species and metabolites for design of resource competition against C. difficile. (A) Overall project schematic of model-guided community design. The design space is comprised of commensal species and C. difficile preferred resources. The phylogenetic tree of the species design space is composed of 29 highly prevalent and diverse species, generated by multiple sequence alignment on the 16S region according to sequencing results. Species color indicates phylum. Species two-letter abbreviations are noted in brackets next to species names. The resource design space consists of 11 resources, all major substrates previously shown to be used by C. difficile during in vivo colonization. The design-test-learn (DTL) cycles were applied for sufficient exploration of the species and metabolite design space. Bayesian experimental design is applied on the consumer resource model to identify experiments to constrain model parameter uncertainty. Given a set of nₛ species and nm resources, the proposed consumer resource model is a set of ordinary differential equations. The DTL cycles terminate when predictive performance saturates for the consumer resource model. The entire dataset from all DTL cycles were used for model-guided community designs. Two models were employed separately: a consumer resource model focusing on resource competition mechanism, and a gradient boosting model capturing all interaction modalities. Communities designed by each model were tested for in vitro C. difficile inhibition, in vitro metabolite consumption, and in vivo C. difficile prevention. (B) Clustered heatmap of monoculture fitness of human gut commensals in 1 g/L single carbohydrate media. The 10 single carbohydrate media are defined media with 1 g/L of each of the ten carbohydrates from the metabolite design space. A no carbohydrate media condition is included to study the effect of amino acids on species growth. The monoculture fitness is determined by the area under the curve (AUC) of 24-hour growth curves. The heatmap is clustered using the Seaborn Clustermap module. Species colors represent phylum. Target species C. difficile (CD) is bolded and highlighted in black. Asterisks represent statistical significance compared to No Carb condition: *P<0.05, **P<0.01, ***P<0.001 according to an unpaired t-test.

FIG. 2. Bayesian model-guided exploration of species-resource landscape improve model prediction of species abundance and provide biological insights into factors effecting C. difficile abundance. (A) The bottom-up community assembly workflow for testing the C. difficile inhibition of various species combinations in different metabolite combinations. The species and metabolites tested in each experimental condition are either rationally designed in DTL0 or generated from Bayesian optimization for maximum information gain. Synthetic communities are cultured in 96-well deep well plates in anaerobic conditions and incubated at 37 °C. At various timepoints, the communities are sampled for species absolutes abundance, as determined by total cell density (OD600) and species relative abundance. Species relative abundances are determined using multiplexed 16S rRNA sequencing. (B) Scatterplot of measured 24 hr C. difficile OD versus predicted 24 hr C. difficile OD of consumer resource in 10-fold cross validation. 10-fold cross validation is performed on monoculture and community growth dynamics from all DTL cycles. Prediction performances are evaluated by Pearson correlation and mean squared error between measured and predicted 24 hr C. difficile OD. Datapoints represent average over biological replicates (2-3). Due to the number of unique experimental conditions, individual replicates are omitted from the figure. (C) Clustered heatmap of consumer resource model prediction performance across DTL cycles. Prediction performances are evaluated by Pearson correlation between predicted and experimental species abundance in 10-fold cross-validation. The number of unique experimental conditions in each DTL cycle is labeled in grey. Species colors represent phylum. Target species C. difficile (CD) is bolded and highlighted in black. (D) Multidimensional scaling (MDS) principal coordinate plots of all DTL and validation community data. Experimental conditions are represented by binary vectors that capture presence or absence of species phylogeny and metabolites. Phylogenetic information is augmented into experimental condition vectors based on parent nodes in the phylogenetic tree. The resulting experimental design is a 68-dimensional vector that captures presence or absence of the 29 species (encoded in 57 phylogenetic node vectors) and 11 metabolites. We performed multidimensional scaling (MDS) using a Euclidean distance metric to project the experimental design onto 2-dimensions using Scikit-Learn's MDS function⁵⁷. Each point represents a unique experimental condition. Colors represent DTL cycle or validation (left) and 24 hr C. difficile OD600 (right). Marker edge color in the right plot represents communities further validated in in vitro exo-metabolomics and murine model. Only conditions inoculated with C. difficile are included in the right plot.

FIG. 3. Model interpretation and biological insights from the consumer resource model (CR-comm) and the gradient boosting model (GB). (A) Clustered heatmap of conversion coefficients in the CR-comm model. The CR-comm model is trained on all DTL data including both monoculture and community growth dynamics. The heatmap is clustered using the Seaborn Clustermap module. Species colors represent phylum. Target species C. difficile (CD) is bolded and highlighted in black. (B) Gradient boosting model schematic. The model is trained on both monoculture and community growth dynamics from all DTL cycles. The inputs for the model are inoculum species abundance and metabolite concentrations. The output from the model is 24-hour C. difficile OD. The model is coupled with interpretable machine learning method SHAP analysis to compute SHAP values to quantify contributions from each factor on 24-hour C. difficile OD. (C) Scatterplots of measured 24 hr C. difficile OD versus predicted 24 hr C. difficile OD of gradient boosting model in 10-fold cross validation. 10-fold cross validation is performed on monoculture and community growth dynamics from all DTL cycles. Prediction performances are evaluated by Pearson correlation and mean squared error between measured and predicted 24 hr C. difficile OD. (D), I SHAP value distribution of species (D) and metabolites I. SHAP values are calculated for all experimental conditions where a given feature (species or metabolite) is present. Each datapoint represent feature contribution in a unique experimental condition. Features are sorted by mean SHAP value from negative to positive. Colors indicate normalized inoculum species OD600 (D) or normalized inoculum metabolite concentration I calculated from dividing experimental inoculum concentration by the maximum inoculum concentration across the entire dataset.

FIG. 4. The in vitro validation of C. difficile inhibition by model-designed communities. (A) Schematic of model-designed community design and validation. All DTL experimental data were used to fit CR model and GB model, separately. Each model was used to perform in silico prediction of 24 hour C. difficile OD in all 4-member communities in 22 media conditions. The 22 media conditions (as illustrated by the heatmap) include full carbohydrates and leave-one-out carbohydrate conditions (1 g/L for each carbohydrate) at two different amino acid levels (5 g/L or 10 g/L). For higher C. difficile inhibition across metabolic environments, communities are ranked by maximum C. difficile OD across all in silico media conditions (as illustrated by the heatmap), with the lowest maximum C. difficile OD communities selected for validation. Fifteen communities with the lowest maximum C. difficile OD from each model were selected for validation (CR group and GB group). Fifteen communities with the highest maximum C. difficile OD from both models were also selected for validation (Weak group). Additionally, fifteen random communities were tested in validation (Random group). (B) Distribution of experimental 24-hour C. difficile OD across all 22 media conditions. Each distribution includes a total of 330 unique experimental conditions from 15 communities in 22 media conditions. Asterisks represent statistical significance: *P<0.05, **P<0.01, ***P<0.001 according to an unpaired t-test.

FIG. 5. Normalized metabolite consumption by C. difficile and model-designed communities. (A) Bipartite network of normalized metabolite consumption by C. difficile and model-designed communities. Model-designed communities include two four-member communities designed by gradient boosting model (GB1 and GB2) and two four-member communities designed by consumer resource model (CR1 and CR2). Species were cultured in media containing 1 g/L of all 10 target carbohydrates with 10 g/L amino acids. At the start of culture, supernatants were harvested for measurements of metabolite concentrations by LC-MS (Time 0 sample). After 24 hours of growth, cell pellets were collected for contamination screening by NGS sequencing of 16S region and supernatants were harvested for measurement of metabolite concentrations by LC-MS (Time 24 sample). Total depletion of each metabolite is determined by subtracting concentration in Time 0 sample by concentration in Time 24 sample. Normalized consumption is determined by subtracting total depletion in each experimental condition by the total depletion in media blank to account for loss due to evaporation and degradation. The edge thickness represents the average of normalized metabolite consumption (Mm) for each metabolite in each experimental condition (n=3). Statistical significances compared to media blanks are determined by p-values according to an unpaired t-test. Only edges with a magnitude>0 and p-value<0.05 are shown. Colors represent C. difficile monoculture or model-designed communities. (B), (C) Barplots of normalized consumption of galactose and mannose (B) and cellobiose and trehalose (C) by C. difficile monoculture and model-designed communities. Bars represent average of normalized metabolite consumption for each metabolite in each experimental condition, and markers represent normalized metabolite consumption in each individual biological replicates (n=3). Asterisks represent statistical significance compared to media blank: *P<0.05, **P<0.01, ***P<0.001 according to an unpaired t-test.

FIG. 6. Model-designed communities confer diverse host responses and colonization in murine models. (A) Schematic of the murine experiment testing different model-designed communities. Mice were tagged and placed on defined diet 3 days before oral gavage of human gut communities (GB1, GB2, CR1, CR2, Max, or no gavage for Cdonly). After 1 week of community colonization, 2000 CFU of C. difficile was introduced by oral gavage. After 2 weeks, mice were sacrificed and cecal contents collected. Mice were weighed and fecal samples were collected every two to three days. Fecal and cecal samples are characterized via C. difficile colony forming units (CFU) counting and multiplexed 16S rRNA sequencing. Cecal samples were characterized via C. difficile toxin ELISA assay. (B) Normalized mouse weight after C. difficile challenge. Colors represent experimental groups. Transparent lines represent biological replicates (each mouse) and solid lines represent average over all biological replicates (4 for GB1, GB2, CR1, and CR2, and 5 for Cdonly and Max). Asterisks represent statistical significance compared to Max and colors represent the group compared: *P<0.05, **P<0.01, ***P<0.001 according to an unpaired t-test (FIG. 20 (A)). (C) C. difficile CFU per microliter of fecal sample. Colors represent experimental groups. Transparent lines represent biological replicates (each mouse) and solid lines represent average over all biological replicates (4 for GB1, GB2, CR1, and CR2, and 5 for Cdonly and Max). Dashed line represents limit of detection. Each biological replicate is average over 3 technical replicates. Asterisks represent statistical significance compared to Max and colors represent the group compared: *P<0.05, **P<0.01, ***P<0.001 according to an unpaired t-test (FIG. 20 (B). (D) C. difficile relative abundance in fecal and cecal sample. Colors represent experimental groups. Transparent lines represent biological replicates (each mouse) and solid lines represent average over all biological replicates (4 for GB1, GB2, CR1, and CR2, and 5 for Max). Asterisks represent statistical significance compared to Max and colors represent the group compared: *P<0.05, **P<0.01, ***P<0.001 according to an unpaired t-test (FIG. 20 (C)).

FIG. 7. Monoculture growth in single carbohydrate media, related to FIG. 1. (A), (B) Clustered heatmap of monoculture fitness of human gut commensals in 5 g/L (A) or 10 g/L (B) single carbohydrate media. The 10 single carbohydrate media are defined media with 5 g/L (A) or 10 g/L (B) of each of the ten carbohydrates from the metabolite design space. A no carbohydrate media condition is included to study the effect of amino acids on species growth. The monoculture fitness is determined by the area under the curve (AUC) of 24-hour growth curves. The heatmap is clustered using the Seaborn Clustermap module. Species colors represent phylum. Target species C. difficile (CD) is bolded and highlighted in black. Asterisks represent statistical significance compared to No Carb condition: *P<0.05, **P<0.01, ***P<0.001 according to an unpaired t-test. (C) Clustered heatmap of conversion coefficients in the consumer resource model of monoculture growth dynamics. The heatmap is clustered using the Seaborn Clustermap module. Species colors represent phylum. Target species C. difficile (CD) is bolded and highlighted in black. (D) Jaccard index of resource utilization overlap between C. difficile and each species in CR-mono model. The Jaccard index is calculated by the number of resources utilized by both C. difficile and the species of interest divided by the total number of resources. Resources with a Cᵢⱼ conversion coefficient at least two standard deviations from 0 are classified as resources utilized by a species. Species are ranked by Jaccard index from low to high. Species colors represent phylogeny.

FIG. 8. Data from nutrient perturbation experiments in DTL0, related to FIG. 2. (A) Violin plot of C. difficile abundance by resource in media. 20 different media conditions were investigated, including single substrate dropouts at two different amino acid concentrations. Species ODs were sampled at 24 hour and were determined by total cell density (OD600) and species relative abundance. Species relative abundances are determined using multiplexed 16S rRNA sequencing. Each datapoint is a unique experimental condition averaged over 2-3 replicates. Asterisks represent statistical significance: *P<0.05, **P<0.01, ***P<0.001 according to an unpaired t-test. (B) Violin plot of C. difficile abundance by species in the inoculum community in DTL0. Each violin represents the distribution of C. difficile absolute abundance in experimental conditions where a given species is present in the community. Species ODs were sampled at 24 hour and were determined by total cell density (OD600) and species relative abundance. Species relative abundances are determined using multiplexed 16S rRNA sequencing. Each datapoint is a unique experimental condition averaged over 2-3 replicates. Species are sorted from low to high median C. difficile absolute abundance. The Jaccard index with C. difficile from CR-mono model for high overlap species is marked under species names, specifically 0.82 for BO and BY and 0.72 for EC, BC, BT, EL, BU, and PJ.

FIG. 9. Experimental conditions in each DTL cycle and high C. difficile abundance cluster, related to FIG. 2. (A), (B) Heatmap of model-selected species (A) and metabolite (B) experimental conditions in each DTL cycle. Occurrence is determined by the count of experimental conditions in each DTL cycle that includes a given species (A) or metabolite (B). All species combinations of monoculture and 2-member, 3-member, and 4-member communities were included in the design algorithm for each DTL cycle. For computational efficiency and feasibility, DTL1, DTL2, DTL3 each included 5 out of 10, 5 out of 10, and 6 out of 10 carbohydrates in the design algorithm, respectively. (C) Consumer resource model prediction performance of C. difficile abundance evaluated by mean squared error (MSE) across DTL cycles. MSE are calculated between predicted and experimental species abundance in 10-fold cross-validation. (D) Multidimensional scaling (MDS) principal coordinate plots of all DTL and validation community data colored by cluster assignments. Kmeans clustering is performed on experimental conditions represented by binary vectors that capture presence or absence of species phylogeny and metabolites, using Scikit-Learn's Kmeans function. I, (F) Plots of sum squared error I and average silhouette score (F) used to select number of clusters. Sum squared error is obtained from the inertia of Scikit-Learn's Kmeans function. Average silhouette score is obtained using Scikit-Learn's Silhouette Score function. (G) Bar plot of inoculum species counts from DTL cycle experimental conditions in the high C. difficile abundance cluster. Experimental conditions from all DTL data in cluster 4 are selected for inoculum species occurrence counting. (H) Violin plot of C. difficile abundance by species in the inoculum community in all DTL data. Each violin represents the distribution of C. difficile absolute abundance in experimental conditions where a given species is present in the community. Species ODs were sampled at 24 hour and were determined by total cell density (OD600) and species relative abundance. Species relative abundances are determined using multiplexed 16S rRNA sequencing. Each datapoint is a unique experimental condition averaged over 2-3 replicates. Species are sorted from low to high median C. difficile absolute abundance. The Jaccard index with C. difficile from CR-comm model for select species of interest is marked under species names, specifically 0.09 for BH, 0.18 for EH, 0.27 for EC, and 0.36 for EL.

FIG. 10. Comparison between CR and CR-mono parameters and prediction performance of a modified linear regression model, related to FIG. 3. (A) Clustered heatmap of log ratio of conversion coefficients between CR model and CR-mono model. Color indicates higher conversion coefficient in CR model (red) or CR-mono model (blue). The heatmap is clustered using the Seaborn Clustermap module. (B) Jaccard index of resource utilization overlap between C. difficile and each species in CR model. The Jaccard index is calculated by the number of resources utilized by both C. difficile and the species of interest divided by the total number of resources. Resources with a Cᵢⱼ conversion coefficient at least two standard deviations from 0 are classified as resources utilized by a species. Species are ranked by Jaccard index from low to high. Species colors represent phylogeny. (C) Scatterplots of measured 24 hr C. difficile OD versus predicted 24 hr C. difficile OD of modified linear regression model in 10-fold cross validation. 10-fold cross validation is performed on monoculture and community growth dynamics from all DTL cycles. Prediction performances are evaluated by Pearson correlation and mean squared error between measured and predicted 24 hr C. difficile OD. One outlier at (0.38, −47935.51) is not depicted on the scatterplot.

FIG. 11. Model predictions for in vitro validation communities, related to FIG. 4. (A), (B) CR (A) and GB (B) predicted 24 hr C. difficile OD distribution of CR-opt, GB-opt, Random, and Low-Inhibition groups. Distributions represent predicted 24 hr C. difficile OD across 22 in silico media conditions.

FIG. 12. Species counts in in vitro validation communities, related to FIG. 4. (A), (B), (C), (D) Species counts in 15 optimized communities in CR-opt (A), GB-opt (B), random (C), or low-inhibition set designed by CR and GB (D). The 15 optimized communities designed by CR or GB were designed by minimizing the maximum 24 hr C. difficile OD across 22 in silico media conditions. The random communities were designed by the Numpy Random package. The low-inhibition communities were designed by maximizing the minimum 24 hr C. difficile OD across 22 in silico media conditions in both CR and GB predictions.

FIG. 13. Model prediction performances of in vitro validation communities, related to FIG. 4. (A), (B) Scatterplots of measured 24 hr C. difficile OD versus predicted 24 hr C. difficile OD of CR model (A) and GB model (B) for in vitro validation. Colors indicate validation group identity. Prediction performances are evaluated by Pearson correlation and mean squared error between measured and predicted 24 hr C. difficile OD. The prediction Pearson correlation for Random group by CR is 0.42, with p-value of 1.79*10⁻⁹. The prediction Pearson correlation for Random group by GB is 0.58, with p-value of 2.30*10⁻¹⁸.

FIG. 14. Predictions and measured 24 hr C. difficile OD distributions for exo-metabolomic and mouse experiment communities, related to FIG. 5. (A), (B), (C) Distribution of maximum 24 hr C. difficile OD in CR-opt communities (A), GB-opt communities (B), and low-inhibition communities designed by CR and GB (C). The maximum C. difficile OD for each community is determined from the maximum 24 hr C. difficile OD across in vitro validations in 22 media conditions. The maximum C. difficile OD for communities selected for metabolomics and murine experiments are marked by vertical lines (CR1, CR2, GB1, GB2, and Max). (D) Measured in vitro 24 hr C. difficile OD distribution of communities selected for exo-metabolomics and murine model experiment across 22 media conditions. Each datapoint represent average over 3 biological replicates.

FIG. 15. Species relative abundance in model-designed communities and leave-one-out communities, related to FIG. 5. (A)-(D) Stacked barplots of relative species relative abundances in model-guided communities GB1 (A), GB2 (B), CR1 (C), CR2 (D) and their leave-one-out subcommunities. Species were cultured in media containing 1 g/L of all 10 target carbohydrates with 10 g/L amino acids. After 24 hr of growth, cell pellets were collected for contamination screening by NGS sequencing of 16S region. Relative abundances for each species are calculated based on 16S NGS sequencing result, dividing species read counts by total read counts. Bar colors indicate species identity. Each experimental condition contains two or three biological replicates.

FIG. 16. Normalized metabolite consumption by C. diff, model-designed communities, and leave-one-out communities, related to FIG. 5. (A)-(H) Barplots of normalized consumption of leucine (A), proline (B), succinate (C), galactose and mannose (D), sorbitol and mannitol I, GlcNAc and GalNAc (F), sialic acid (G), and cellobiose and trehalose (H) by C. difficile monoculture, model-designed communities, and leave-one-out communities. Model-designed communities include two four-member communities designed by gradient boosting model (GB1 and GB2) and two four-member communities designed by consumer resource model (CR1 and CR2). Leave-one-out communities are three-member communities based on model-designed communities with one species left out. Species were cultured in media containing 1 g/L of all 10 target carbohydrates with 10 g/L amino acids. At the start of culture, supernatants were harvested for measurements of metabolite concentrations by LC-MS (Time 0 sample). After 24 hr of growth, cell pellets were collected for contamination screening by NGS sequencing of 16S region and supernatants were harvested for measurement of metabolite concentrations by LC-MS (Time 24 sample). Total depletion of each metabolite is determined by subtracting concentration in Time 0 sample by concentration in Time 24 sample. Normalized consumption is determined by subtracting total depletion in each experimental condition by the total depletion in media blank to account for loss due to evaporation and degradation. Bars represent average of normalized metabolite consumption for each metabolite in each experimental condition, and markers represent normalized metabolite consumption in each individual biological replicates (n=3). Asterisks represent statistical significance compared to media blank: *P<0.05, **P<0.01, ***P<0.001 according to an unpaired t-test.

FIG. 17. Clustered heatmap of normalized metabolite fold changes in by C. difficile monoculture, model-designed communities, and leave-one-out communities, related to FIG. 5. Model-designed communities include two four-member communities designed by gradient boosting model (GB1 and GB2) and two four-member communities designed by consumer resource model (CR1 and CR2). Leave-one-out communities are three-member communities based on model-designed communities with one species left out. Species were cultured in media containing 1 g/L of all 10 target carbohydrates with 10 g/L amino acids. At the start of culture, supernatants were harvested for measurements of metabolite concentrations by LC-MS (Time 0 sample). After 24 hr of growth, cell pellets were collected for contamination screening by NGS sequencing of 16S region and supernatants were harvested for measurement of metabolite concentrations by LC-MS (Time 24 sample). Absolute fold change of each metabolite is determined by dividing area-under-the-curve in Time 24 sample by area-under-the-curve in Time 0 sample. Normalized fold change is determined by dividing absolute fold change in each experimental condition by the absolute fold change in media blank to account for loss due to evaporation and degradation. Colors represent average normalized fold change over 3 biological replicates. Darker blue represents lower normalized fold change and higher consumption. Subcommunities with EC left out are marked by a star. Asterisks represent statistical significance compared to media blank: *P<0.05, **P<0.01, ***P<0.001 according to an unpaired t-test.

FIG. 18. Species relative abundance barplots of murine experiment, related to FIG. 6. Species relative abundance barplot of C. difficile challenge mouse experiment with GB1 (A), GB2 (B), CR1 (C), CR2 (D), and Max I, respectively. Relative abundances for each species are calculated based on 16S NGS sequencing result, dividing species read counts by total read counts. Bar colors indicate species identity. Each experimental condition contains five biological replicates (each mouse) for Max and four biological replicates (each mouse) for GB1, GB2, CR1, and CR2.

FIG. 19. Mouse survival and cecal C. difficile toxin concentration, related to FIG. 6. (A) Percentage of mouse survival over time. Colors represent experimental group. (B) C. difficile toxin A and toxin B total concentration in cecal sample. Cecal C. difficile toxin concentration is measured by ELISA. Colors represent groups and markers represent individual biological replicates (each mouse). Asterisks represent statistical significance: *P<0.05, **P<0.01, ***P<0.001 according to an unpaired t-test.

FIG. 20. Statistical significance of murine experimental data, related to FIG. 6. (A), (B), (C) The p-value between experimental groups measurements of normalized mouse weight (A), C. difficile CFU/UI in fecal and cecal sample (B), and C. difficile relative abundance in fecal and cecal sample (C) over time. The p-values are calculated by unpaired t-test.

### DETAILED DESCRIPTION OF THE INVENTION

Disclosed herein are compositions comprising bacterial communities. The bacterial communities can comprise purified bacterial strains. In some embodiments, the compositions are in the form of pharmaceutical compositions comprising the bacterial communities. In some embodiments, the compositions are in the form of food products comprising the bacterial communities. Also disclosed are methods of treating a pathogenic infection, such as Clostridium difficile (C. difficile) infection, in a subject by administering the compositions to the subject.

Various factors including antibiotic usage can induce dysbiosis of the gastrointestinal tract, which may allow for colonization by pathogenic microorganisms, such as C. difficile. Such colonization or pathogenic infection can lead to a variety of adverse effects in the subject including diarrhea, which is one of the primary symptoms characteristic of C. difficile infection (CDI). In the case of CDI, diarrhea is thought to be a result of C. difficile production of Toxin B (also referred to as cytotoxin TcdB), which results in opening of the tight junctions between intestinal epithelial cells, increasing vascular permeability, hemorrhage, and inflammation.

The compositions described herein are effective in the treatment of C. difficile infection. As shown herein, the disclosed compositions are effective in suppressing the pathogenic effects of C. difficile infection. The compositions provided herein can reduce the amount of C. difficile after infection and thereby provide an effective method for eliminating C. difficile from the body (e.g., the gut). The compositions disclosed herein have been found to reduce or inhibit production or activity of C. difficile Toxin B and thereby represent effective compositions for the treatment or prevention of CDI. The compositions disclosed herein have also been found to inhibit the growth and/or survival of C. difficile.

The present disclosure provides compositions comprising bacterial communities of purified bacterial strains that can be administered to subjects experiencing or having experienced a pathogenic infection to treat the infection. In some embodiments, the compositions may be administered to subjects who may be at risk for a pathogenic infection. Such subjects include subjects who previously had pathogenic infections, subjects who have been treated with antibiotics, and subjects who will undergo a procedure that will put them at an increased risk for a pathogenic infection (e.g., surgery and/or hospitalization). In some embodiments, the pathogenic infection is infection by a pathogen that is present predominantly in the gut or the intestine. In some embodiments, the pathogen that is present predominantly in the gut or the intestine is Clostridium difficile. In some embodiments, the one or more of the bacterial strains of the compositions provided herein colonize or recolonize the intestinal tract or parts of the intestinal tract (e.g., the colon or the cecum) of the subject. Such colonization or recolonization may also be referred to as grafting. In some embodiments, the one or more of the bacterial strains of the compositions recolonize the intestinal tract (e.g., the colon or the cecum) of the subject after the naturally present microbiome has been partially or completely removed, e.g., because of administration of antibiotics. In some embodiments, the one or more of the bacterial strains of the compositions colonize a dysbiotic gastrointestinal tract.

In some embodiments, the one or more of the bacterial strains of the compositions can “outgrow” a pathogen, such as C. difficile. Thus, in some embodiments, if a pathogen (e.g., C. difficile) and one or more bacteria of compositions provided herein are both present in the intestinal tract (e.g., the colon or the cecum), the one or more bacteria of compositions provided herein grow faster (e.g., have a shorter doubling time) than the pathogen, thereby preventing the pathogen from accumulating in the intestinal tract (e.g., the colon or the cecum). In some embodiments, the faster growth results because the one or more bacteria of the compositions provided herein are better at grafting in the intestinal tract (e.g., the colon or the cecum). In some embodiments, the faster growth results because the one or more bacteria of the compositions provided herein are better at metabolizing nutrients present in the intestinal tract (e.g., the colon or the cecum). In some embodiments, the compositions of bacterial strains provided herein prevent or inhibit production of bacterial toxins by the pathogenic infection, or prevent or inhibit the cytopathic or cytotoxic effects of such bacterial toxins.

In some embodiments, the bacterial strains of the compositions provided herein can treat pathogenic infections because of the synergy between the bacterial strains. Thus, without being limiting, in some embodiments, the combination of the bacterial strains of the compositions provided herein act synergistically because the combination of the strains is particularly well-suited to use nutrients in the intestinal tract (e.g., the colon or the cecum), for instance through metabolic interactions, and/or because the combination is superior in grafting (e.g., by providing a favorable microenvironment).

In some embodiments, a pathogenic infection such as C. difficile is treated because the combination of bacterial strains of the compositions provided herein is superior in the use of nutrients when compared to the pathogen such as C. difficile, thereby suppressing the growth of the pathogen such as C. difficile. In some embodiments, a pathogenic infection such as C. difficile is treated because the combination of bacterial strains of the compositions provided herein is superior in grafting when compared to the pathogen such as C. difficile, thereby suppressing the growth of the pathogen such as C. difficile. In some embodiments, a pathogenic infection such as C. difficile is treated because the combination of bacterial strains of the compositions provided herein is superior in the use of nutrients and in grafting when compared to the pathogen such as C. difficile, thereby suppressing the growth of the pathogen such as C. difficile. In some embodiments, a pathogenic infection such as C. difficile is treated because the combination of bacterial strains of the compositions provided herein inhibits the growth and/or survival of the pathogen such as C. difficile. In some embodiments, a pathogenic infection such as C. difficile is treated because the combination of bacterial strains of the compositions provided herein inhibits the growth and/or survival of the pathogen and induces regulatory T cells (Tregs) in the subject that results in reduction or elimination of the pathogen such as C. difficile.

In some embodiments, the synergistic effect is provided by the capacity of the combination to colonize specific niches in the intestinal tract (e.g., the colon or the cecum). In some embodiments, the synergistic effect is provided by the capacity of the combination to metabolize specific nutrients. In some embodiments, the synergistic effect is provided by the capacity of the combination to provide specific metabolites to the environment. Such specific metabolites may suppress growth of the pathogen and/or stimulate growth of non-pathogens. In some embodiments, the synergistic effect is provided by the capacity of the combination to provide short-chain fatty acids to the environment. In some embodiments, the synergistic effect is provided by the capacity of the combination to provide specific short-chain fatty acids to the environment. In some embodiments, the synergistic effect is provided by the capacity of the combination to produce butyrate. In some embodiments, the synergistic effect is provided by the capacity of the combination to produce acetate. In some embodiments, the synergistic effect is provided by the capacity of the combination to produce lactate. In some embodiments, the synergistic effect is provided by the capacity of the combination to produce propionate. In some embodiments, the synergistic effect is provided by the capacity of the combination to produce succinate. In some embodiments, the synergistic effect is provided by the capacity of the combination to produce multiple metabolites. In some embodiments, the synergistic effect is provided by the capacity of the combination to produce multiple short-chain fatty acids. In some embodiments, the synergistic effect is provided by the capacity of the combination to produce both butyrate and acetate. In some embodiments, the synergistic effect is provided by the capacity of the combination to produce both butyrate and lactate. In some embodiments, the synergistic effect is provided by the capacity of the combination to produce both butyrate and propionate. In some embodiments, the synergistic effect is provided by the capacity of the combination to produce both butyrate and succinate. In some embodiments, the synergistic effect is provided by the capacity of the combination to produce butyrate, acetate and additional short-chain fatty acids.

In some embodiments, the bacterial strains used in the compositions provided herein are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals). In some embodiments, the compositions comprise bacterial strains purified from different sources. The bacterial strains purified from different sources can be combined together after being purified from their respective sources. The bacterial strains purified from different sources can be grown before being combined together. The bacterial strains purified from different sources can include any two, any three, any four, any five, any six, any seven, or more of the bacterial strains described herein as being included in the compositions of the invention.

In one aspect, the disclosure provides compositions comprising bacterial communities of one or more bacterial strains.

The one or more bacterial strains can comprise a strain of any one or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of: Escherichia coli (EC); and any two or more or any three or more of Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of: Escherichia coli (EC); Bacteroides uniformis (BU); and any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

In some embodiments, the bacterial community comprises a purified bacterial strain of each of: Escherichia coli (EC); Bacteroides uniformis (BU); and any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

In some embodiments, the bacterial community comprises a purified bacterial strain of each of: Escherichia coli (EC); and any one or more, any two or more, or each of Bacteroides uniformis (BU), Bacteroides fragilis (BF), and Roseburia intestinalis (RI).

In some embodiments, the bacterial community comprises a purified bacterial strain of each of Escherichia coli (EC), Bacteroides uniformis (BU), Bacteroides fragilis (BF), and Roseburia intestinalis (RI).

“A” purified bacterial strain as used herein can comprise or consist of one purified strain or multiple purified bacterial strains from each listed type (e.g. species).

In some embodiments, the bacterial community can be considered to consist of individual bacterial cells of the specified purified bacterial strains, to thereby distinguish the individual cells of the specified purified bacterial strains from cells of other bacterial strains that may or may not be present in the composition. In various embodiments, a number of the individual bacterial cells in the bacterial community is at least 1%, at least 5%, at least 10%, at least 15%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% (by number) of the total number of individual bacterial cells in the composition.

In some embodiments, one or more types (e.g., species) of bacteria can be considered to be “excluded” from the compositions of the invention. “Excluded” as used herein in this context refers to one or more defined types of bacteria (i.e., the excluded bacteria) constituting no more than a specified proportion of the total number of individual bacterial cells in a given composition. In various versions of the invention, the excluded types of bacterial constitute no more than 50%, no more than 45%, no more than 40%, no more than 35%, no more than 30%, no more than 25%, no more than 20%, no more than 15%, no more than 10%, no more than 5%, no more than 1%, no more than 0.5%, no more than 0.1%, no more than 0.05%, no more than 0.01%, no more than 0.005%, no more than 0.001%, no more than 0.0005%, no more than 0.0001%, no more than 0.00005%, or no more than 0.00001% of a total number of individual bacterial cells in the composition.

In various embodiments, excluded bacteria include strains of any one or more of Acidaminococcus fermentans, Acidaminococcus intestine, Anaerostipes hadrus, Anaerotruncus colihominis DSM 17241, Anaerotruncus colihominis, Bifidobacterium bifidum, Blautia coccoides, Blautia faecis, Blautia hansenii, Blautia luti, Blautia producta ATCC 27340, Blautia producta, Blautia wexlerae, Clostridium bacterium UC5.1-1D4, Clostridium bolteae 90A9, Clostridium bolteae, Clostridium disporicum, Clostridium hathewayi, Clostridium hiranonis, Clostridium innocuum, Clostridium orbiscindens 1_3_50AFAA, Clostridium symbiosum WAL-14163, Clostridium symbiosum, Dracourtella massiliensis GDI, Dracourtella massiliensis, Eisenbergiella tayi, Erysipelatoclostridium ramosum, Erysipelotrichaceae_bacterium_21_3, Eubacterium contortum, Eubacterium fissicatena, Eubacterium rectale, Flavinofractor plautii, Fusicatenibacter saccharivorans, Lachnospiraceae bacterium 7-_I_58FAA, Lactobacillus mucosae, Marvinbryantaformexigens, Megasphaera elsdenii, Pseudoflavinofractor capillosus, Roseburia faecis, Ruminococcus albus, Ruminococcus champanellensis, Ruminococcus faecis, Ruminococcus obeum, Ruminococcus torques, Sellimonas intestinalis, Subdoligranulum, Turicibacter sanguinis, Parabacteroides johnsonii (PJ), Phocaeicola vulgatus (PV), Bacteroides cellulosilyticus (BY), Enterocloster asparagiformis (CG), Anaerobutyricum hallii (EH), Anaerostipes caccae (AC), Agathobacter rectalis (ER), Peptacetobacter hiranonis (CH), Eggerthella lenta (EL), Bifidobacterium pseudocatenulatum (BP), Bifidobacterium longum (BL), Bifidobacterium adolescentis (BA), Bacteroides thetaiotaomicron (BT), Bacteroides ovatus (BO), Bacteroides caccae (BC), Collinsella aerofaciens (CA), Coprococcus comes (CC), Faecalibacterium prausnitzii (FP), Segatella copri (PC), Dorea formicigenerans (DF), Blautia hydrogenotrophica (BH), Clostridium scindens (CS), Dorea longicatena (DL), Desulfovibrio piger (DP), Clostridium hiranonis, Bacteroides thetaiotaomicron, Bacteroides uniformis, Phocaeicola vulgatus, Clostridium scindens, Collinsella aerofaciens, and/or Desulfovibrio piger, in any combination.

In some embodiments, excluded bacteria include the combination of strains of Acidaminococcus fermentans, Acidaminococcus intestine, Anaerostipes hadrus, Anaerotruncus colihominis DSM 17241, Anaerotruncus colihominis, Bifidobacterium bifidum, Blautia coccoides, Blautia faecis, Blautia hansenii, Blautia luti, Blautia producta ATCC 27340, Blautia producta, Blautia wexlerae, Clostridium bacterium UC5.1-1D4, Clostridium bolteae 90A9, Clostridium bolteae, Clostridium disporicum, Clostridium hathewayi, Clostridium hiranonis, Clostridium innocuum, Clostridium orbiscindens 1_3_50AFAA, Clostridium symbiosum WAL-14163, Clostridium symbiosum, Dracourtella massiliensis GDI, Dracourtella massiliensis, Eisenbergiella tayi, Erysipelatoclostridium ramosum, Erysipelotrichaceae_bacterium_21_3, Eubacterium contortum, Eubacterium fissicatena, Eubacterium rectale, Flavinofractor plautii, Fusicatenibacter saccharivorans, Lachnospiraceae bacterium 7-_I_58FAA, Lactobacillus mucosae, Marvinbryantaformexigens, Megasphaera elsdenii, Pseudoflavinofractor capillosus, Roseburia faecis, Ruminococcus albus, Ruminococcus champanellensis, Ruminococcus faecis, Ruminococcus obeum, Ruminococcus torques, Sellimonas intestinalis, Subdoligranulum, and Turicibacter sanguinis.

In some embodiments, excluded bacteria include the combination of strains of Parabacteroides johnsonii (PJ), Phocaeicola vulgatus (PV), Bacteroides cellulosilyticus (BY), Enterocloster asparagiformis (CG), Anaerobutyricum hallii (EH), Anaerostipes caccae (AC), Agathobacter rectalis (ER), Peptacetobacter hiranonis (CH), Eggerthella lenta (EL), Bifidobacterium pseudocatenulatum (BP), Bifidobacterium longum (BL), and Bifidobacterium adolescentis (BA).

In some embodiments, excluded bacteria include the combination of strains of Bacteroides thetaiotaomicron (BT), Bacteroides ovatus (BO), Bacteroides caccae (BC), Collinsella aerofaciens (CA), Coprococcus comes (CC), Faecalibacterium prausnitzii (FP), Segatella copri (PC), Dorea formicigenerans (DF), Blautia hydrogenotrophica (BH), Clostridium scindens (CS), Dorea longicatena (DL), and Desulfovibrio piger (DP).

In some embodiments, excluded bacteria include the combination of strains of Clostridium hiranonis, Bacteroides thetaiotaomicron, Bacteroides uniformis, Phocaeicola vulgatus, Clostridium scindens, Collinsella aerofaciens, and Desulfovibrio piger.

In some embodiments, the compositions described herein comprise spore forming and non-spore forming bacterial strains. In some embodiments, the compositions described herein comprise spore forming bacterial strains. In some embodiments, the compositions described herein comprise only spore forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore form may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form (as discussed above, spore forming bacteria can also be in vegetative form). In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

In some embodiments, the disclosure provides compositions wherein the compositions comprise bacterial strains that are spore forming bacterial strains. In some embodiments, the disclosure provides compositions wherein the compositions comprise bacterial strains that are non-spore forming bacterial strains. In some embodiments, the disclosure provides compositions wherein the compositions comprise bacterial strains that are spore forming bacterial strains and bacterial strains that are non-spore forming bacterial strains. In some embodiments, the disclosure provides compositions, wherein the compositions comprise a mixture of bacterial strains wherein at least 10% of the bacterial strains are spore forming bacterial strains, at least 20% of the bacterial strains are spore forming bacterial strains, at least 30% of the bacterial strains are spore forming bacterial strains, at least 40% of the bacterial strains are spore forming bacterial strains, at least 50% of the bacterial strains are spore forming bacterial strains, at least 60% of the bacterial strains are spore forming bacterial strains, at least 70% of the bacterial strains are spore forming bacterial strains, at least 80% of the bacterial strains are spore forming bacterial strains, at least 90% of the bacterial strains are spore forming bacterial strains. In some embodiments, up to 100% of the bacterial strains are spore forming bacterial strains. Whether a bacterial strain is a spore forming strain can be determined for instance by evaluating the genome of the bacterial strain for the presence of sporulation genes. However, it should be appreciated that not all bacteria that are predicted to encode spore forming genes can be made to sporulate. In addition, whether a bacterial strain is a spore forming strain can be determined by exposing the bacterial strain to stress conditions, e.g., heat or exposure to chemicals (e.g., ethanol or chloroform), that are known to induce sporulation.

In some embodiments of the compositions provided herein, the spore forming bacteria are in spore form. In some embodiments of the compositions provided herein, the spore forming bacteria are in vegetative form. In some embodiments of the compositions provided herein, the spore forming bacteria are both present in spore form and in vegetative form. In some embodiments, the disclosure provides compositions, wherein the compositions comprise spore forming bacteria at least 10% of the spore forming bacteria are in spore form, at least 20% of the spore forming bacteria are in spore form, at least 30% of the spore forming bacteria are in spore form, at least 40% of the spore forming bacteria are in spore form, at least 50% of the spore forming bacteria are in spore form, at least 60% of the spore forming bacteria are in spore form, at least 70% of the spore forming bacteria are in spore form, at least 80% of the spore forming bacteria are in spore form, at least 90% of the spore forming bacteria are in spore form, up to 100% in spore form.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

Methods of inducing sporulation of spore-forming bacterial strains are well known in the art (See e.g., Paredes-Sabja et al., Trends Microbiol. (2011) 19(2):85-94). Generally, bacterial strains that are spore-formers can be made to go into spore form by stressing the bacterial strains. Non-limiting examples of stresses that can induce sporulation are an increase in temperature, change in the nutrients available and/or exposure to chemicals (e.g., ethanol or chloroform). It should be noted that bacteria that are non-spore formers, for instance because they are missing sporulation genes, cannot be made to sporulate by stress. To prepare compositions in which all the bacterial strains are in the spore form, the composition or bacterial cultures used to prepare the composition may be subjected to treatment to kill any bacteria not in spore form (e.g., in vegetative form), for example by exposing the composition to heat and are chemically breaking down the non-spore bacteria. The bacteria in spore form can subsequently be separated from the non-spore bacteria for instance by filtration.

The amount of spores can be quantified using techniques known in the art. These techniques include phase contrast microscopy for enumerating spores using a hemocytometer. In addition, the viability of spores can be determined by plating the spores and growing the spores. For instance, spores can be plated in appropriate media and incubated in the anaerobic chamber for a period of time (e.g., 48-96 hrs.). Viability can subsequently be determined by quantifying the colony forming units which correspond to spores that germinated. For instance, spores can be plated on TCCFA plates (Taurocholate, cycloserine, cefoxintin, fructose agar plates), in which taurocholate helps the spores to germinate. In addition, spores can be quantified using the dipicolinic assay (DPA assay). DPA is an agent that allows for spore selection and is a clear indicator of endospores. When complexed with terbium, bright green luminescence is observed.

In any of the compositions provided herein, in some embodiments, the bacterial strains are purified. In any of the compositions provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein can be isolated from the microbiome of healthy individuals. However, bacterial strains can also be isolated from individuals that are considered not to be healthy. In some embodiments, the compositions include strains originating from multiple individuals or sources.

As used herein, the term “isolated” bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

Aspects of the present disclosure are related to methods for treating a pathogenic infection in a subject by administering a therapeutically effective amount of any of the compositions described herein. In some embodiments, the subject is a mammalian subject, such as a human, non-human primate, rodent, rabbit, sheep, pig, dog, cat, horse, or cow. In some embodiments, the subject is a human subject. In some embodiments, the subject is a carrier of a pathogenic organism and is suffering from the effects of the infection (e.g., diarrhea caused by C. difficile toxins). In some embodiments the subject is an asymptomatic carrier of a pathogen. In some embodiments, the subject is a carrier of C. difficile. In some embodiments the subject is an asymptomatic C. difficile carrier. In some embodiments, the subject has experienced recurrent or chronic pathogenic infections. In some embodiments, the subject is suffering from a first occurrence of a particular pathogenic infection. In some embodiments, the subject has been treated with antibiotics which resulted in the recurrence of the pathogenic infection. In some embodiments, the subject has been treated with antibiotics which resulted in a first occurrence of a pathogenic infection. In some embodiments, the subject is to undergo a procedure that puts the subject at a higher risk of infection. In some embodiments, the compositions provided herein are administered to a subject to lower the risk of becoming infected by a pathogen.

In some embodiments, the compositions provided herein are administered to a subject if the subject has a dysbiosis (e.g., has as microbiome associated with a disease state). In some embodiments, treatment with the compositions provided herein results in the change in the microbiome of the subject. In some embodiments, treatment with the compositions provided herein removes the dysbiosis in the subject resulting in a healthy microbiome. In some embodiments, treatment with the compositions provided herein removes the dysbiosis in the subject resulting in microbiome refractory or less susceptible to infection by a pathogen.

As used herein, the term “pathogen” in regard to a pathogenic infection refers to a microorganism (e.g., a bacterium) that causes a disease or a disease state in a subject. In some embodiments, the disease or disease state of the subject may include symptoms such as colitis, diarrhea, watery diarrhea, abdominal cramping, fever, blood or pus in the stool, nausea, dehydration, loss of appetite, chills, weight loss, and/or kidney failure. In some embodiments, the pathogenic infection may be diagnosed, for example, by detecting a pathogen (or protein or nucleic acid associated with a pathogen) in a fecal sample collected from the subject. In some embodiments, the pathogenic infection may be diagnosed, for example, by comparing the microbiota of a fecal sample of the subject with the microbiota in a fecal sample of a healthy subject.

In some embodiments, the pathogenic infection is C. difficile. In some embodiments, the C. difficile is an antibiotic-resistant C. difficile, e.g., fluoroquinolone resistant C. difficile.

Additional non-limiting examples of pathogens responsible for pathogenic infection that can be treated according to the methods provided herein can include Clostridium perfringens, Clostridium botulinum, Clostridium tributrycum, Clostridium sporogenes, Escherichia coli, Pseudomonas aeruginosa, such as multidrug-resistant Pseudomonas aeruginosa, vancomycin-resistant Enterococci (VRE) carbapenem-resistant Enterobacteriaceae (CRE) Neisseria gonorrheae, Acinetobacter, multidrug-resistant Acinetobacter, Campylobacter, multidrug-resistant Campylobacter, Candida, Fluconazole-resistant Candida, extended spectrum beta-lactamase (ESBL) producing Enterobacteriaceae, Salmonella, Salmonella Typhimurium, drug-resistant non-typhoid Salmonella spp., drug-resistant Salmonella Typhi, drug-resistant Shigella, Staphylococcus aureus, such as methicillin-resistant S. aureus or vancomycin-resistant S. aureus, drug-resistant Streptococcus pneumoniae, drug-resistant Tuberculosis, erythromycin-resistant Group A Streptococcus, clindamycin-resistant Group B Streptococcus, vancomycin-resistant Enterococci, and any combination thereof. Additional non-limiting examples of pathogens responsible for pathogenic infection that can be treated according to the methods provided herein can include Leishmania, Staphylococcus epidermis, Staphylococcus saprophyticus, Streptococcus pyogenes, Streptococcus pneumoniae, Streptococcus agalactiae, Enterococcus faecalis, Corynebacterium diptheriae, Bacillus anthracis, Listeria monocytogenes, Clostridium perfringens, Clostridium tetanus, Clostridium botulinum, Clostridium difficile, Neisseria meningitidis, Neisseria gonorrhoeae, Escherichia coli, Salmonella typhimurium, Salmonella cholerasuis, Salmonella enterica, Salmonella enteriditis, Yersinia pestis, Yersinia pseudotuberculosis, Yersinia enterocolitica, Vibrio cholerae, Campylobacter jejuni, Campylobacter fetus, Helicobacter pylori, Pseudomonas aeruginosa, Pseudomonas mallei, Haemophilus influenzae, Bordetella pertussis, Mycoplasma pneumoniae, Ureaplasma urealyticum, Legionella pneumophila, Treponema pallidum, Leptospira interrogans, Borrelia burgdorferi, Mycobacterium tuberculosis, Mycobacterium leprae, Chlamydia psittaci, Chlamydia trachomatis, Chlamydia pneumoniae, Rickettsia ricketsii, Rickettsia akari, Rickettsia prowazekii, Brucella abortus, Brucella melitens, Brucella suis, Francisella tularensis, and any combination thereof. In general, any bacterium that is capable of inducing a disease in a subject and/or that is not present in healthy individual is considered a pathogen herein. It should be appreciated that a subject may carry multiple pathogens and/or have multiple pathogenic infections.

Any of the compositions described herein may be administered to a subject in a therapeutically effective amount or a dose of a therapeutically effective amount to treat or prevent a pathogenic infection (e.g., one or more pathogenic infections). The terms “treat” or “treatment” refer to reducing or alleviating one or more of the symptoms associated with a pathogenic infection, reducing the amount of bacterial toxin produced by the pathogenic infection, and/or reducing the bacterial load of the pathogenic infection. The terms “prevent” or “prevention” encompass prophylactic administration and may reduce the incidence or likelihood of pathogenic infection or a recurrent or chronic pathogenic infection. For instance, in some embodiments, administration of the compositions provided herein result in a healthy microbiome that is refractory to pathogenic infection, thereby preventing the pathogenic infection.

As used herein, a “therapeutically effective amount” of a composition, such as a pharmaceutical composition, is any amount that results in a desired response or outcome in a subject, such as those described herein, including but not limited to prevention of infection, an immune response or an enhanced immune response to the pathogenic infection, prevention or reduction of symptoms associated with pathogenic infection, and/or a reduction or inhibition of toxin production by the pathogenic infection. It should be appreciated that the term “effective amount” may be expressed in number of bacteria or bacterial spores to be administered. It should further be appreciated that the bacteria can multiply once administered. Thus, administration of even a relatively small amount of bacteria may have therapeutic effects.

In some embodiments, the therapeutically effective amount of any of the compositions described herein is an amount sufficient to enhance survival of the subject, reduce the bacterial burden of the pathogenic infection in the subject, and/or reduce or inhibit toxin production by the pathogenic infection. In some embodiments, the therapeutically effective amount is an amount sufficient to reduce the bacterial burden of the pathogenic infection in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 105-fold or more, as compared to the bacterial burden in a subject with a pathogenic infection that has not received any of the compositions described herein, or as compared to a fecal sample from the same subject that was collected prior to administration of any of the compositions.

In some embodiments, the compositions provided herein inhibit the production of a bacterial toxin, e.g., C. difficile Toxin B. In some embodiments, the therapeutically effective amount is an amount sufficient to reduce or inhibit the amount of bacterial toxin (e.g., C. difficile Toxin B) produced by pathogenic infection in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of the bacterial toxin in a subject with a pathogenic infection that has not received any of the compositions described herein or as compared to a fecal sample from the same subject that was collected prior to administration of any of the compositions.

In some embodiments, the therapeutically effective amount is an amount sufficient to recolonize or repopulate the gastrointestinal tract of the subject with non-pathogenic bacteria. In some embodiments, the therapeutically effective amount is an amount sufficient to graft one or more of the bacterial strains of the composition in the gastrointestinal tract of the subject. In some embodiments, a fecal sample is obtained from the subject to assess the bacterial burden of the pathogenic infection and/or evaluate the efficacy of administration of the bacterial compositions described herein. In some embodiments, the microbiota of the subject (e.g., the identity and abundance of strains and/or species of the microbiota) may be assessed to determine a disease state of the subject and/or assess progress of the treatment. In some embodiments, the microbiota of the subject having a pathogenic infection is compared to the microbiota of a healthy subject, such as a subject that is not experiencing or has not experienced the pathogenic infection. In some embodiments, the microbiota of the subject having a pathogenic infection is compared to the microbiota of the same subject from a fecal sample obtained from the subject prior to the pathogenic infection.

Any of the compositions described herein, including the pharmaceutical compositions and food products comprising the compositions (see below), may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. See, e.g., U.S. Pat. Nos. 3,261,761, 4,205,132, WO 2014/029578, and WO 2012/098358, incorporated herein by reference in their entireties. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may subsequently be combined with a pharmaceutical excipient. In some embodiments, the bacterial strain is a lyophilized cake. In some embodiments, the compositions comprising the one or more bacterial strains are a lyophilized cake.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the invention can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the bacteria are formulated for delivery to the intestines (e.g., the small intestine and/or the colon). In some embodiments, the bacteria are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech, (2016) 17 (1), 56-67).

The bacteria may also be formulated for rectal delivery to the intestine (e.g., the colon). Thus, in some embodiments, the bacterial compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include Ph sensitive compositions, more specifically, buffered sachet formulations or enteric polymers that release their contents when the Ph becomes alkaline after the enteric polymers pass through the stomach. When a Ph sensitive composition is used for formulating the pharmaceutical preparation, the Ph sensitive composition is preferably a polymer whose Ph threshold of the decomposition of the composition is between about 6.8 and about 7.5. Such a numeric value range is a range in which the Ph shifts toward the alkaline side at a distal portion of the stomach, and hence is a suitable range for use in the delivery to the colon. It should further be appreciated that each part of the intestine (e.g., the duodenum, jejunum, ileum, cecum, colon, and rectum) has a different biochemical and chemical environment. For instance, parts of the intestines have different pHs, allowing for targeted delivery by compositions that have a specific Ph sensitivity. Thus, the compositions provided herein may be formulated for delivery to the intestine or specific parts of the intestine (e.g., the duodenum, jejunum, ileum, cecum, colon, and rectum) by providing formulations with the appropriate Ph sensitivity. (See e.g., Villena et al., Int J P harm 2015, 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers. The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as Ph specific coatings, allow for the delivery of the compositions provided herein to a desired target area (e.g., the cecum or the colon).

The compositions comprising bacterial strains can be formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration., e.g. as a suppository. In some embodiments, the pharmaceutical composition is formulated for delivery to the intestine or a specific area of the intestine (e.g., the colon) by providing an appropriate coating (e.g., a Ph specific coating, a coating that can be degraded by target area specific enzymes, or a coating that can bind to receptors that are present in a target area).

Dosages of the active ingredients in the pharmaceutical compositions of the present invention can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of a pathogenic infection, reduction of bacterial burden of pathogenic infection, reduction or inhibition of toxin production) is achieved. In general, effective doses of the compositions of the present invention, for the prophylactic treatment of groups of people as described herein vary depending upon many different factors, including routes of administration, physiological state of the subject, whether the subject is human or an animal, other medications administered, and the therapeutic effect desired. In some embodiments, the dosing regimen entails oral administration of a dose of any of the compositions described herein. In some embodiments, the dosing regimen entails oral administration of multiple doses of any of the compositions described herein. In some embodiments, the composition is administered orally to the subject once, twice, 3 times, 4 times, 5 times, 6 times, 7 times, 8 times, 9 times, or at least 10 times.

The compositions, including the pharmaceutical compositions disclosed herein, can include a range of active ingredients (e.g., live bacteria, bacteria in spore form). The amount of bacteria in the compositions may be expressed in weight, number of bacteria and/or CFUs (colony forming units).

In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacteria of the composition per dosage amount. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that the bacteria of the compositions may be present in different amounts. Thus, for instance, as a non-limiting example, a composition may include 10³ of bacteria A, 10⁴ of bacteria B and 10⁶ of bacteria C. In some embodiments, the compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacteria in the composition per dosage amount. In some embodiments, the compositions disclosed herein contain about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacteria combined per dosage amount. As discussed above, bacteria of the compositions may be present in different amounts. In some embodiments, the compositions disclosed herein contain about 10⁻⁷, about 10⁻⁶, about 10⁻⁵, about 10⁻⁴, about 10⁻³, about 10⁻², about 10⁻¹ or more grams of each of the bacteria in the composition per dosage amount. In some embodiments, the compositions disclosed herein contain about 10⁻⁷, about 10⁻⁶, about 10⁻⁵, about 10⁻⁴, about 10⁻³, about 10⁻², about 10⁻¹ or more grams in total for all of the bacteria combined per dosage amount. In some embodiment, the dosage amount is one administration device (e.g., one table, pill or capsule). In some embodiment, the dosage amount is the amount that is administered in a particular period (e.g., one day or one week).

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacteria of the composition per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹ between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacteria of the composition per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹¹, between 10⁴ and 10¹¹, between 10⁵ and 10¹¹, between 10⁶ and 10¹¹, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

In some embodiments, the compositions disclosed herein contain between 10⁻⁷ and 10¹, between 10⁻⁶ and 10¹, between 10⁻⁵ and 10¹, between 10⁻⁴ and 10¹, between 10⁻³ and 10¹, between 10⁻² and 10¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of each of the bacteria in the composition per dosage amount.

In some embodiments, the compositions disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of all of the bacteria combined per dosage amount.

Also with the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the amounts of bacteria (e.g., by weight, amount or CFU) as described above. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

In some embodiments, the subject has not received a dose of an antibiotic prior to administration of the bacterial composition. In some embodiments, the subject has not been administered an antibiotic at least 1, at least 2, at least 3, at least 5, at least 10, at least 15, at least 20, at least 25, at least 30, at least 60, at least 90, at least 120, at least 180 or at least 360 days prior to administration of the compositions provided herein. In some embodiments, the person has not been administered an antibiotic to treat the pathogenic infection. In some embodiments, the compositions provided herein comprise the first treatment of the pathogenic infection.

In some embodiments, the subject may be administered one or more doses of an antibiotic prior to or concurrently with a bacterial composition. Generally, the first line of defense in the treatment of a pathogenic infection is the administration of an antibiotic. In some embodiments, the subject is administered a single dose of an antibiotic prior to the bacterial composition. In some embodiments, the subject is administered multiple doses of an antibiotic prior to the bacterial composition. In some embodiments, the subject is administered at least 2, 3, 4, 5 or more doses of an antibiotic prior to the bacterial composition. In some embodiments, the subject is administered a dose of an antibiotic at substantially the same time as the bacterial composition. Examples of antibiotics that can be administered include, without limitation, kanamycin, gentamicin, colistin, metronidazole, vancomycin, clindamycin, fidaxomicin, and cefoperazone.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Human Gut Communities for Robust C. difficile Inhibition

### Summary

Resource competition plays a crucial role in shaping the fitness and colonization ability of Clostridioides difficile; however, its impact across diverse environmental contexts remains poorly understood. To address this, we leverage the strengths of mechanistic modeling and machine learning (ML) combined with high-throughput, bottom-up community construction to investigate resource competition's influence on C. difficile growth in vitro and within the mammalian gut. Using a design-test-learn framework, Bayesian experimental design guided the characterization of thousands of combinations of 29 microbial species and 11 key resources critical to C. difficile's metabolic niche. Guided by the ML and mechanistic model, we identified microbial communities capable of robustly inhibiting C. difficile across diverse nutrient environments. ML-optimized communities demonstrated superior inhibitory performance compared to mechanistic model-optimized communities in vitro. Notably, an ML-optimized community with a unique metabolic niche and overall metabolomics profile similar to the profile of C. difficile reduced C. difficile to undetectable levels in the murine gut. This integrated experimental and computational approach provides a foundation for uncovering driving mechanisms and designing communities to robustly inhibit C. difficile colonization.

### Introduction

The human gut microbiome shifts dynamically between a healthy and a dysbiotic state due to the tug-of-war between commensals and pathogens. Clostridioides difficile (C. difficile or CD) is an opportunistic bacterial pathogen that can infect the human gastrointestinal tract, leading to C. difficile infection (CDI). Antibiotics, the standard first-line treatment for CDI, are often associated with recurrent infections (rCDI) due to their disruption of the gut microbiome 1,2. While fecal microbiota transplantation (FMT) has shown efficacy in treating rCDI, its reliance on donor microbiota introduces challenges such as inconsistent patient outcomes and potential transfer of antibiotic-resistant organisms³⁻⁵. Overcoming these challenges, certain defined microbial consortia have been evaluated in clinical trials and approved for CDI treatment over the past decade^{6,7}. These consortia are often identified via screening of reduced complexity communities with the desired functions from healthy donors⁸ as opposed to being designed based on mechanistic insights. Our lack of understanding of the mechanisms of action or failure modes of bacterial therapeutics constrains their therapeutic potential.

Resource competition is a key mechanism that shapes ecological dynamics and microbial colonization of the human gut. Therefore, this mechanism modality has potential for impacting pathogens such as C. difficile^{9,10}. While the metabolic niche of C. difficile has not been comprehensively mapped, C. difficile has been shown to occupy distinct metabolic niches during infection as the microbiome composition was varied¹¹. For example, several metabolites have been shown to enhance C. difficile colonization and toxicity¹², including amino acids^{10,13}, mucus-derived sugars¹⁴, sorbitol¹⁵, succinate¹⁶, trehalose¹⁷, mannitol ^{18,19}, and cellobiose²⁰. As a proof of concept, a community of mucus-derived sugar utilizers competing with C. difficile reduced colonization²¹. Corroborating the role of resource competition influencing C. difficile disease severity, an avirulent strain of C. difficile protected mice from virulent C. difficile-induced colitis via amino acid depletion²². Consistent with these results, non-toxigenic C. difficile strains protected hamsters from infection²³. Therefore, resource competition is a major mechanism influencing C. difficile colonization.

Bottom-up synthetic community assembly coupled with computational modeling²⁴ can be leveraged to explore combinatorial landscapes of species and environmental factors. Considering variability in these factors is critical to understanding C. difficile colonization due to inter-individual variability and intra-individual dietary shifts and other environmental factors^{25,26}. Further, C. difficile may exploit different metabolic niches over time due to its metabolic plasticity^{27,28}. The data generated using high-throughput culturing of microbial communities can inform computational models to predict their dynamics and decipher interactions²⁹. For example, the consumer resource model (CR), a dynamic mechanistic model that captures metabolite-mediated interactions³⁰, has been used to decipher mechanistic insights^{31,32}. Since the synthetic microbial community design spaces grow exponentially with the number of input variables, Bayesian experimental design^{33,34} can allow efficient exploration of the large species and resource design space.

Mechanistic models are based on known interaction modes shaping community dynamics. By contrast, machine learning methods can learn to capture complex microbial interaction mechanisms from data³⁵. Among machine learning methods, models based on the construction of an ensemble of regression trees have been shown to excel at the prediction of microbial community data³⁶⁻³⁹. For example, Light Gradient Boosting Model (LightGBM or GB)^{40,41} is a tree-based model that displays improved prediction performance with skewed or heavily tailed feature distributions compared to other machine learning models such as neural networks⁴⁰, a common feature of microbial community datasets. To uncover interactions shaping microbial communities, interpretable artificial intelligence techniques such as SHapley Additive explanation (SHAP) using the trained models can elucidate the contributions of input variables on the model's output prediction⁴¹⁻⁴³.

Leveraging high-throughput bottom-up synthetic microbial community assembly and model-guided experimental design, we investigated C. difficile growth in human gut communities across different nutrient environments. We constructed a species design space of 29 human gut commensals and a resource design space of 11 metabolites critical for C. difficile's metabolic niche in the mammalian gut. To explore the large design space (240 considering presence/absence of variables), we used design-test-learn (DTL) cycles coupled with a consumer resource model and Bayesian experimental design to generate an information-rich dataset of 2432 unique conditions. To evaluate resource competition as a design rule for C. difficile inhibition, we evaluated differences in inferred interactions and model prediction performance using a flexible machine learning (ML) model and a mechanistic consumer resource (CR) model. Communities optimized for C. difficile inhibition across a wide range of nutrient environments were selected for validation in vitro and in a murine model. Our results demonstrate that both models identified communities that robustly inhibited C. difficile growth across nutrient environments in vitro. In a murine model, a ML-designed community displayed a unique metabolite utilization profile of key resources in vitro, substantially reduced C. difficile to undetectable levels and reduced disease severity. This implies that mechanisms not captured by the consumer resource model may influence C. difficile in the mammalian gut. In sum, our framework which integrates high-throughput bottom-up community assembly, mechanistic modeling and machine learning, establishes a foundation for the discovery of design principles of community-level interactions impacting human pathogens.

### Results

### Overview of the Model-Guided Design-Test-Learn Approach to Design Space Exploration

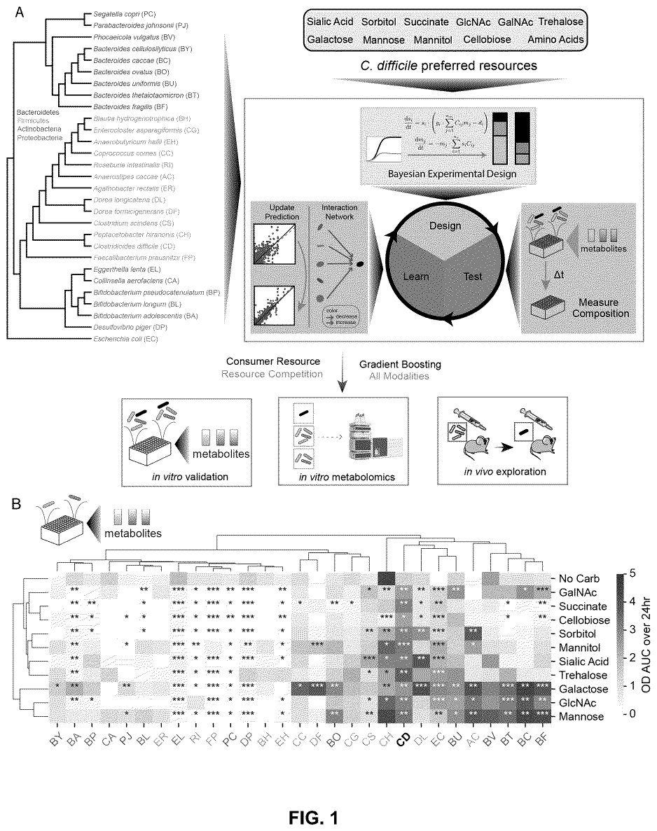

To investigate C. difficile inhibition by human gut commensals across diverse nutrient environments, we selected a set of diverse human gut species and metabolites that have been previously shown to be used by C. difficile. In the species design space (FIG. 1 (A)), we included 28 commensal species and C. difficile, consisting of representative members of four major phyla in the human gut microbiome (Bacteroides, Firmicutes, Actinobacteria, and Proteobacteria). The species in this collection are phylogenetically diverse, metabolically diverse⁴⁴, and prevalent across individuals^{45,46}, therefore representative of a healthy human gut microbiome. Many species in the commensal design space have links to C. difficile inhibition. For example, Peptacetobacter hiranonis (CH) and Clostridium scindens (CS) were shown to inhibit C. difficile and ameliorate disease severity in a murine model^{34,47,48}. The resource design space consists of 11 resources previously shown to mediate C. difficile colonization of the murine gut or virulence activities. These resources include amino acids¹³, mucus-derived sugars (acetyl-neuraminic acid (sialic acid), acetyl-glucosamine (GlcNAc), acetyl-galactosamine (GalNAc), galactose, and mannose)¹⁴, sorbitol¹⁵, succinate¹⁶, trehalose¹⁷, mannitol^{18,19}, and cellobiose²⁰.

Due to the immense size of the species and resource design space (~550 billion conditions considering the presence/absence of species and resources), we aimed to efficiently explore the species-resource design space by model-guided DTL cycles (FIG. 1 (A)). To explore the role of resource competition in shaping C. difficile growth, we used a modified CR model capturing resource competition. This model assumes that rate of change of species abundance is strictly coupled with resource consumption and a species-specific death rate. In particular, the key parameter Cᵢⱼ in the CR model represents the consumption rate of resource j by species i. Our framework involves construction of an initial model informed by monoculture and community dynamics in various media conditions. In the second stage, we apply Bayesian experimental design³³ to identify informative conditions by varying the initial abundance of species and concentrations of metabolites expected to minimize model parameter uncertainties (Methods). A previous study demonstrated an inverse relationship between C. difficile abundance and the average richness of human gut communities in vitro⁴⁸. Therefore, we considered communities with low richness (1-5 species) to learn the effects of interactions from the variability in C. difficile across different community contexts. The experimental selected by Bayesian experimental design are characterized in the test phase (150-200 unique conditions). In the learn stage, experimental data are appended to the previous dataset to improve estimates of model parameters and make new predictions of community dynamics. The DTL procedure will terminate when data from new DTL cycles does not substantially improve the model's prediction performance of C. difficile abundance. Overall, the information-rich experimental data can reveal the species and metabolite-mediated interactions shaping C. difficile growth.

Mechanisms beyond resource competition have been shown to impact C. difficile, including antimicrobial production^{49,50}. Therefore, to capture these different interaction modalities, we used a flexible machine learning model (light gradient boosting or GB) to map the initial concentrations of species and metabolites to the endpoint (24 hour) abundance of C. difficile (FIG. 1 (A)). To account for variability in nutrient environments in the mammalian gut^{25,26}, we aimed to identify communities with a fixed richness (4 species) that display consistent C. difficile inhibition across distinct resource environments (i.e. robust anti-C. difficile communities). In the final stage, the CR and GB models were trained on all data collected over DTL cycles to predict C. difficile abundance across all four-member commensal communities across a wide range of media conditions. Communities with lowest maximum C. difficile abundance across all in silico media conditions were selected for in vitro characterization. To investigate the effects of model-optimized communities on C. difficile colonization, we used a germ-free murine model orally gavaged with distinct communities.

Diverse Human Gut Bacteria Display Varying Degrees of Metabolic Niche Overlap with C. difficile

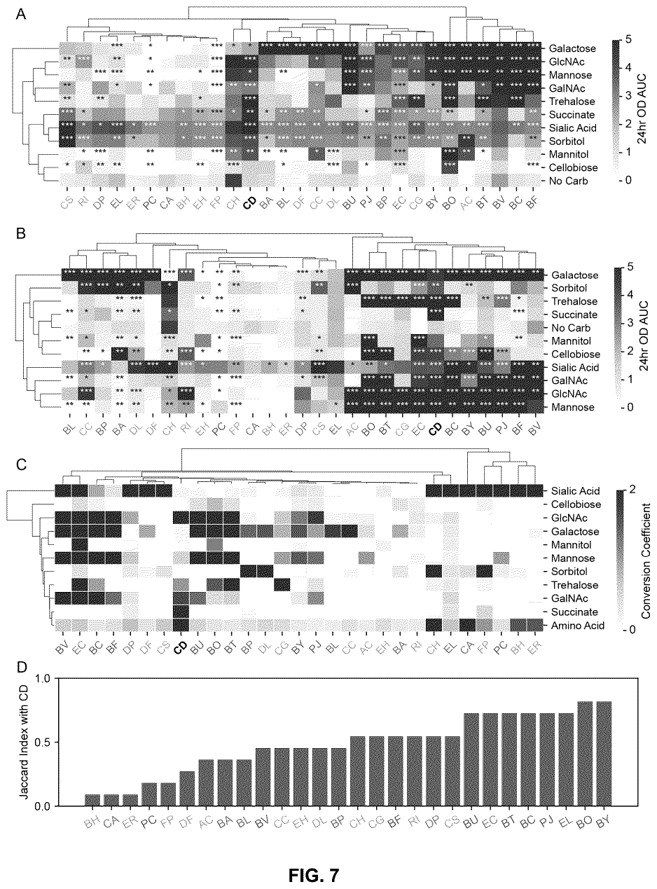

In our initial dataset, we characterized the resource utilization capability of each species in our design space in different nutrient environments to provide deeper insights into metabolic niche overlap with C. difficile and to inform our mechanistic computational model. Our media conditions contained individual metabolites at different concentrations (1 g/L (FIG. 1 (B)), 5 g/L (FIG. 7 (A)), or 10 g/L (FIG. 7 (B))) to uncover the relationship between species growth and metabolite concentrations. Since amino acids can be used as a major energy source for C. difficile⁵¹, we considered with amino acids that lacks other preferred carbon sources (No Carb condition). Species growth was quantified by computing the area-under-the-curve (AUC) over 24 hours based on absorbance at 600 nm (OD600) measurements (899 unique monoculture growth conditions). While C. difficile monoculture displayed growth in the No Carb condition due to Stickland amino acid metabolism, its growth was significantly enhanced in the presence of all single carbon sources (FIGS. 1 (B) and 7 (A, B)). These data demonstrate that all selected resources enhanced C. difficile growth, consistent with the previous studies^{14-20,51}.

The other species displayed growth variation across different nutrient environments. In the presence of 1 g/L supplemented resource, Clostridium scindens (CS), Peptacetobacter hiranonis (CH), Dorea longicatena (DL), and Escherichia coli (EC) displayed significant growth in the presence of multiple carbohydrates compared to the No Carb condition. In addition, Bacteroides species Bacteroides ovatus (BO), Bacteroides uniformis (BU), Bacteroides thetaiotaomicron (BT), Bacteroides caccae (BC), and Bacteroides fragilis (BF) exhibited a significant enhancement in growth in the presence of galactose, GlcNAc, or mannose than the No Carb condition (FIG. 1 (B)). In the presence of higher concentration of resource (5 g/L and 10 g/L), most species (28 of 29 for 5 g/L and 25 of 29 for 10 g/L) displayed significant growth in the presence of multiple carbohydrates compared to No Carb condition (FIG. 7 (A, B)). For the highest resource concentration, species growth profiles clustered based on phylogeny, highlighting that phylogenetic information provides insight into the similarities in functional profiles. In addition, Firmicutes Bifidobacterium longum (BL), Bifidobacterium pseudocatenulatum (BP), and Bifidobacterium adolescentis (BA) displayed similar patterns of galactose and sorbitol utilization. Finally, Bacteroides (BO, BT, BC, BY, BU, PJ, BF, and BV) were clustered based on their utilization of galactose, sialic acid, GalNAc, GlcNAc, and mannose (FIG. 7 (B), consistent with their utilization of mucin-derived sugars⁵²⁻⁵⁴

We fit a CR model to monoculture data (CR-mono) (377 total parameters) to provide insight into the resource utilization capabilities of each species (No Carb and each carbohydrate at three concentration levels). The regularization parameters for minimizing model overfitting (penalizing non-zero parameter values) are estimated using the expectation-maximization algorithm (Methods). Resource utilization of resource j by species i occurs when the mean of Cᵢⱼ is at least two standard deviations from zero, which for a normal distribution indicates 95% of true Cᵢⱼ values will be nonzero⁵⁵. The inferred Cᵢⱼ conversion coefficients indicates that C. difficile uses all resources except sorbitol in monoculture (FIG. 7 (C)). The commensal species were clustered into several groups based on the patterns in the inferred Cᵢⱼ conversion coefficients. For example, a group of species displayed high utilization of mucin-derived sugars (BV, EC, BC, BF). Using Jaccard indexes on the shared resource utilizations determined by Cᵢⱼ parameters, we quantified the metabolic niche overlap between C. difficile and each species. Certain species displayed high predicted metabolic niche overlap with C. difficile (BO and BY) whereas other species exhibited low metabolic niche overlap with C. difficile (BA, CA, and ER) based on monoculture data (FIG. 7 (D). In sum, C. difficile has the capability to utilize all resources in the design space and human gut species displayed distinct resource utilization profiles with varying degrees of overlap with C. difficile.

### Bayesian Experimental Design Explores a Broad Region of the Species-Metabolite Design Space

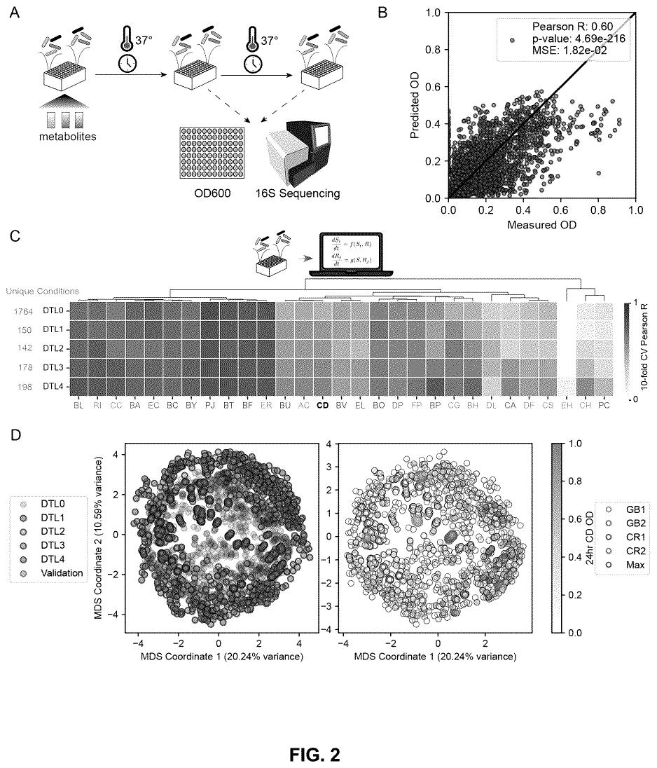

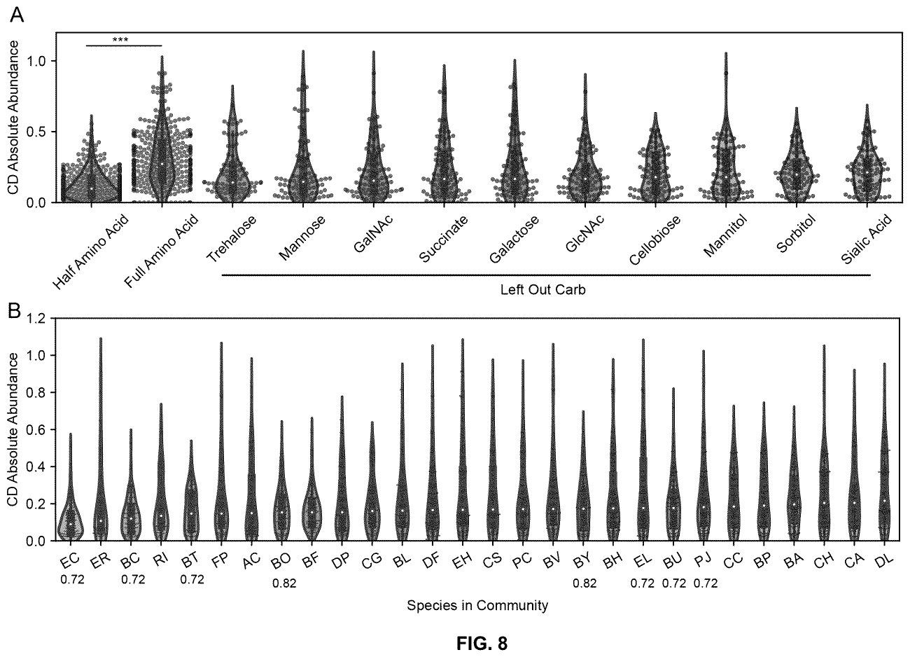

To reveal the patterns of C. difficile growth across the metabolite-species design space, we characterized communities containing C. difficile in a wide range of nutrient environments. Specifically, we tested 22 media conditions, including a mixture of all carbohydrates and all carbohydrates except one (1 g/L for each carbohydrate) in media containing a mixture of amino acids at two distinct concentrations (5 g/L or 10 g/L). We constructed 5-member communities including C. difficile in each media condition and measured species abundances at two time points (16 and 24 hr). The absolute abundance of each species was estimated by computing the product of OD600 and relative abundance based on 16S rRNA gene sequencing^{24,34,48,56} (865 unique conditions) (FIG. 2 (A)). The abundance of C. difficile in four-member communities was significantly enhanced as the amino acid concentration was increased (5 g/L to 10 g/L) (FIG. 8 (A)), consistent with the major role of Stickland metabolism in enabling C. difficile growth. The removal of single carbohydrates did not impact the C. difficile abundance distribution in the context of the mixture (FIG. 8 (A). Further, no single species inhibited C. difficile across all species and resource conditions (FIG. 8 (B)), indicating the absence of a global inhibitor across nutrient environments. Additionally, several species with high monoculture Jaccard index of niche overlap (EC, BO, BC, and BT) (FIG. 7 (D) consistently reduced C. difficile abundance (FIG. 8 (B)), demonstrating that metabolic niche overlap based on monoculture data can provide insight into C. difficile inhibition in microbial communities.

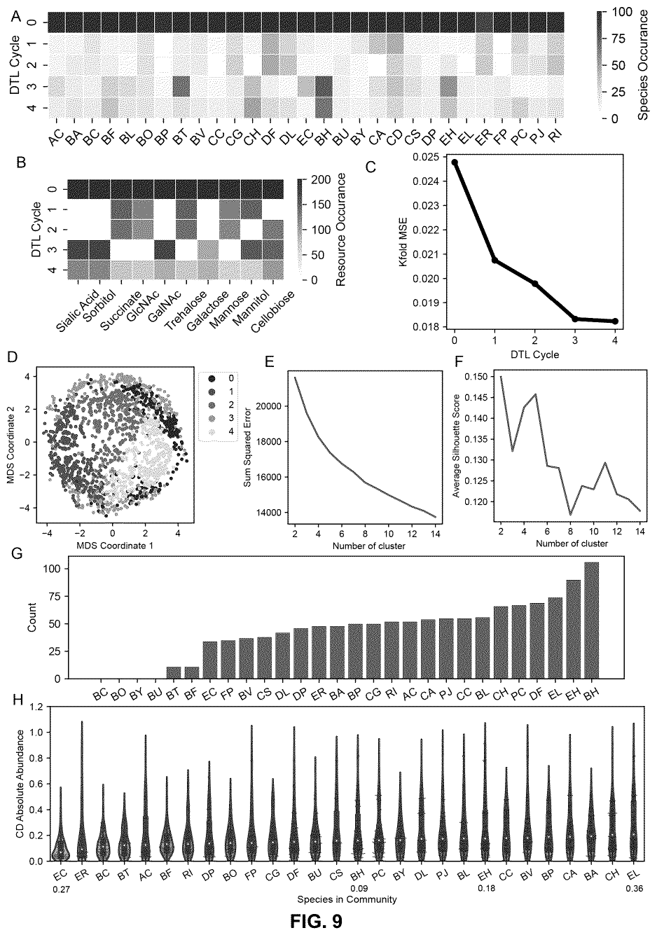

To construct the initial CR model informed by community data (CR-comm), we fit the CR model to a dataset (DTL0) containing 1764 unique experimental conditions, including 865 communities and 899 monocultures. For the CR-comm model, parameters that govern the degree of regularization (penalizing non-zero parameter values) are optimized using the expectation-maximization algorithm (Methods). To improve the model's prediction performance of C. difficile abundance, we used Bayesian experimental design to select combinations of species and metabolites that maximize information content as quantified by the expected Kullback-Leibler divergence between the posterior and prior parameter distributions. Briefly, we fit the model using Bayesian inference to approximate the posterior parameter distribution as a Gaussian. The parameter distribution is then used as a prior to guide experimental designs of species and resource combinations (FIG. 9 (A, B)). We considered 5-6 carbohydrates (10 total) in each round of Bayesian experimental design algorithm due to computational limitations. We prioritized metabolites that were utilized by C. difficile based on the inferred Cᵢⱼ parameters in the CR-comm model (fit to monoculture and community data) in DTL1 (150 unique conditions) and DTL2 (142 unique conditions). In DTL3 (178 unique conditions), we included metabolites underrepresented in previous DTL cycles to further refine our understanding of the metabolite design space. DTL4 (198 unique conditions) was partitioned into a group with metabolites used by C. difficile in communities (succinate, GlcNAc, trehalose and mannitol) and a group with metabolites not used by C. difficile in communities as determined by the Cᵢⱼ parameter values in the CR-comm model (sialic acid, sorbitol, GalNAc, galactose, mannose, and cellobiose). Bayesian experimental design was performed separately on each group.

To evaluate the improvement in model prediction performance across DTL cycles, we used a two-step cross-validation approach. When evaluating prediction performance of the model using data up to a particular DTL cycle, we first perform 10-fold cross-validation on all data collected up to that DTL cycle. In the second step, we train the model on all data up to that DTL cycle and use data from all subsequent DTL cycles as a test set. Combining the held-out predictions from cross-validation and the test predictions from subsequent DTL cycles provides model predictions for all samples in the entire data set. Model prediction performance is evaluated using the Pearson correlation coefficient between model predictions and measured abundances. By using predictions of all samples, we can evaluate how much incorporating data from subsequent DTL cycles improves model prediction performance in a way that ensures the performance metric is consistent across all comparisons. The DTL cycles terminate when the Pearson correlation coefficient of model prediction for C. difficile abundance saturates (<0.1 increase between cycles). Over the four DTL cycles, the Pearson correlation coefficient of C. difficile abundance prediction increased from 0.43 in DTL0 to 0.60 in DTL4 (FIG. 2 (B, C) and the mean squared error of C. difficile abundance prediction decreased from 0.025 in DTL0 to 0.018 in DTL4 (FIG. 9 (C). Beyond C. difficile, 11 human gut species display substantial increase in the Pearson correlation of their predicted abundance across DTL cycles (AC, BP, CC, CG, CH, DF, BH, BU, CA, CS, and PC). In addition, 15 species exhibit high prediction performance based on data from DTL0 (Pearson correlation coefficient >0.5) (BA, BC, BF, BL, BO, BP, BT, CC, EC, BY, DP, ER, FP, PJ, and RI). This implies that the large DTL0 dataset containing community and monoculture experiments is sufficient to accurately predict the abundance of many species. Out of 29 species, 26 display good prediction performance in DTL4 (Pearson correlation coefficient>0.5) except for DL, EH, and PC. In sum, Bayesian experimental design facilitated the efficient exploration of a large metabolite-species design space for building a predictive CR model.

To provide deeper insights into how the Bayesian experimental design algorithm explores the metabolite-species design space across DTL cycles, we used multidimensional scaling (MDS) on community experiments inoculated with C. difficile to visualize the relationships between conditions. The conditions were encoded by binary vectors that capture presence or absence of species, metabolites and the phylogenetic relationships of species. In addition, we can determine if there are patterns in the species and metabolites that map to high or low C. difficile abundance. In each DTL cycle, the algorithm selects experimental conditions on the edge of the design space (FIG. 2 (D)), suggesting that Bayesian experimental design samples extremes within the design space. Across the characterized conditions, many displayed low abundance of C. difficile (FIG. 2 (D)), suggesting that there are potentially many mechanisms that can generate low C. difficile abundance.

A cluster of communities with high C. difficile abundance (cluster 4, mean C. difficile abundance 0.28 OD600, compared to 0.08 for cluster 0, 0.10 for cluster 1, 0.09 for cluster 2, and 0.12 for cluster 3) was identified using K-means clustering (FIG. 9 (D)), with the number of clusters selected based on the sum of squared errors and average silhouette score (FIG. 9 (E, F)). Eggerthella lenta (EL), Eubacterium hallii (EH), and Blautia hydrogenotrophica (BH), spanning two different phyla, were more frequently found in cluster 4 (FIG. 9 (G)). Communities containing EL displayed significantly highest median C. difficile abundance across all DTL data compared to communities containing other species (p-value 2.76*10⁻⁶) (FIG. 9 (H)), This highlights the positive impact of EL on C. difficile that was robust across various species-metabolite environments. By contrast, BH and EH were not among the species with the highest median C. difficile abundances (FIG. 9 (H)), suggesting that BH and EH have context-dependent enhancement of C. difficile growth in the high C. difficile cluster. In sum, our experimental design strategy uncovered a cluster with high C. difficile abundance, revealing key species that can positively impact C. difficile with varying degrees of robustness to species-metabolite perturbations.

Model-Guided Biological Insights into Interactions that Impact Growth of C. difficile

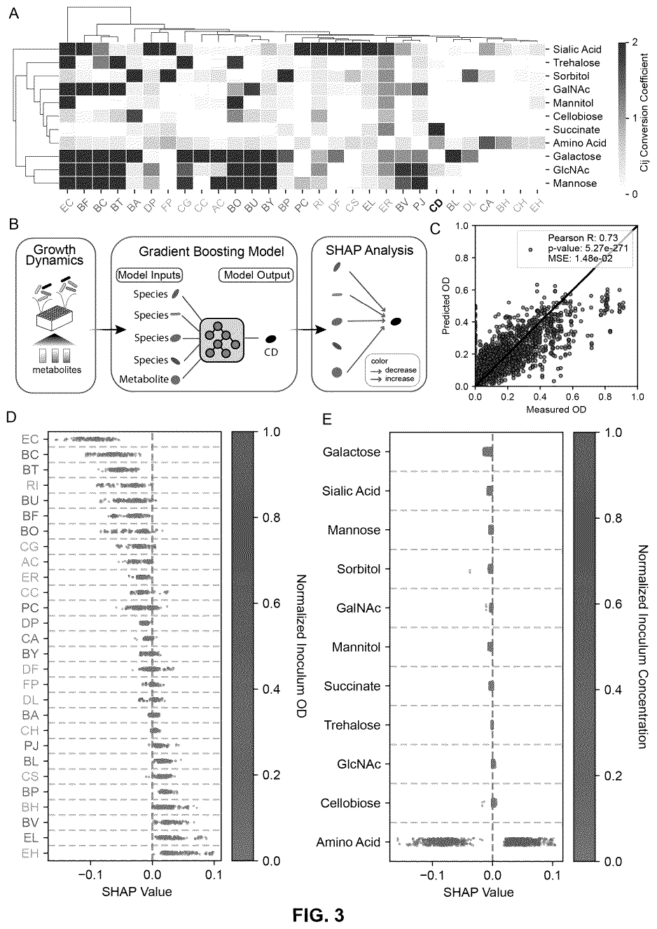

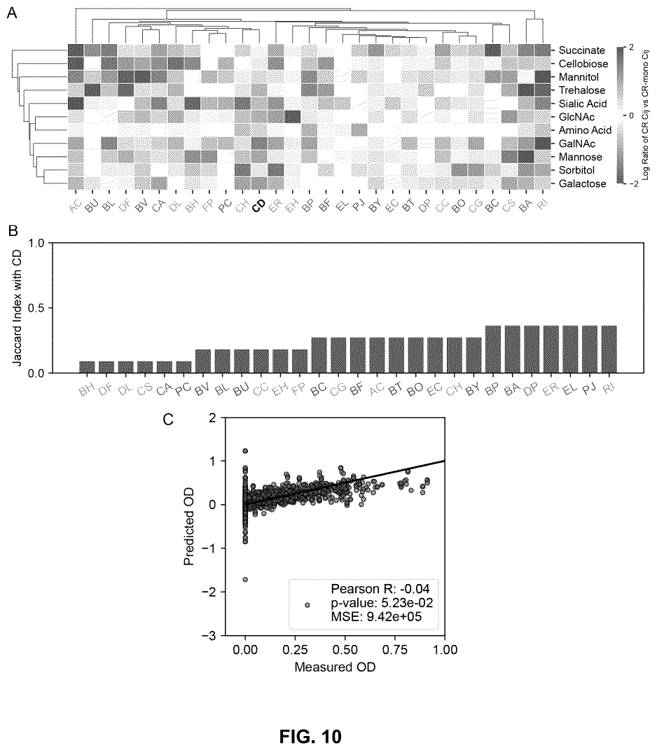

A key advantage of mechanistic models is the ability to examine the parameters directly to gain deeper and causal insights into the interactions driving system behaviors. Therefore, we used a consumer resource model fit to data from all DTL cycles (CR-comm model) to generate biological insights into species-resource interactions. The inferred Cᵢⱼ indicate that C. difficile exhibits high utilization of amino acids, succinate, and GlcNAc in a community context (FIG. 3 (A)). The commensal species cluster into several groups (FIG. 3 (A) including generalists (EC, BF, BC, and BT), specialists that utilize several mucin-derived sugars (CG, CC, AC, BO, BU, and BY), sialic acid (PC, RI, DF, CS, EL, and ER), or other variable patterns. The Cᵢⱼ parameter values for many species changed between the CR-mono model informed by monoculture data and CR-comm model fit to all monoculture and community data (FIG. 10 (A)). Specifically, the Cᵢⱼ parameters for C. difficile for all resources decreased in CR-comm model, suggesting reduced utilization of all resources by C. difficile in community compared to monoculture. The metabolic niche overlap based on the Jaccard index varied for the CR-comm model informed by monoculture versus monoculture and community data (FIG. 10 (B)). In the CR-comm model, BH, DF, DL, CS, CA, and PC are predicted to have low niche overlap with C. difficile whereas BP, BA, DP, ER, EL PJ, and RI are predicted to have high niche overlap with C. difficile. Consistent with a high metabolic niche overlap with EC, C. difficile has the lowest median abundance (FIG. 9 (H)) in communities containing EC. By contrast, communities containing EL displayed the highest median C. difficile abundance (FIG. 9 (H), inconsistent with its high niche overlap with C. difficile (FIG. 10 (B)). This implies that the metabolic niche overlap metric does not always accurately forecast C. difficile abundance.

ML models such as the Light Gradient Boosting Model (GB) mapping the initial species abundance and metabolite concentrations to C. difficile endpoint abundance have the flexibility to capture a wide range of nonlinear and context-dependent interaction modalities. The context-dependent interactions captured by the Light Gradient Boosting Model can be deciphered via explainable artificial intelligence methods like Shapley Additive explanations (SHAP)⁴¹. SHAP calculates the contribution of each input on the model's output. A comparison of the inferred interactions between the CR-comm model (i.e. Cᵢⱼ) and GB model (i.e. SHAP) provide insights into relationships between variables that the consumer resource model fails to capture due to its limited flexibility.

Using all experimental data, we trained a GB model to predict the endpoint (24-hour) C. difficile abundance from the initial species abundances and metabolite concentration (FIG. 3 (B)). The Optuna Python package was used for hyperparameter tuning⁵⁸. The GB model has significantly better prediction performance than a linear regression model with pairwise interactions (FIGS. 3 (C) and 10 (C)), demonstrating that the additional complexity of the GB model improves prediction performance of C. difficile abundance in our system. Further, the prediction performance of the GB model evaluated by 10-fold cross validation is enhanced compared to the CR-comm model (FIGS. 2 (B) and 3 (C)). This highlights that the flexibility of ML can enable improved prediction performance compared to the mechanistic consumer resource model.

SHAP generates a distribution of contributions from the input variable on the model's prediction across all conditions in the dataset. Considering instances where the species or resource is present in the inoculum, a negative SHAP value indicates inhibition of C. difficile by the factor (species or resource), whereas a positive SHAP value indicate enhancement of C. difficile growth. Many species display strong inhibition of C. difficile across all experimental conditions (e.g. EC, BC, BT, RI, BU, BF, and BO) (FIG. 3 (D)). In addition, several species promote C. difficile growth (e.g. PJ, BL, CS, BP, BH, BV, EL, and EH). Galactose inhibits C. difficile (FIG. 3 (E), potentially through promotion of species that inhibit C. difficile (e.g. BC, BT, BC, and BT), consistent with the conversion coefficient analysis in the CR-comm model (FIG. 3 (A)). Across all metabolites, amino acids display the largest magnitude positive impact on C. difficile growth (FIG. 3 (E)), consistent with a significant enhancement of C. difficile growth in DTL0 in the presence of high amino acid concentrations (FIG. 8 (A)).

There are consistent patterns in the inferred interactions in the mechanistic and GB models. For example, EC, BC, BT, RI BF, and BO are predicted to be strong C. difficile inhibitors based on both Cᵢⱼ analysis in CR-comm model (FIG. 10 (B)) and SHAP analysis in GB model (FIG. 3 (D)). In addition, species with low metabolic niche overlap in the CR-comm model (FIG. 10 (B)) promote C. difficile growth in the GB model based on SHAP (BL, BH, and EH) (FIG. 3 (D)). Analysis of both models reveals amino acids as a key resource promoting C. difficile growth. However, there are also certain discrepancies between the two models. While EL is predicted to have high niche overlap with C. difficile based on the CR-comm model (FIG. 10 (B)), it enhances C. difficile growth in the GB model (FIG. 3 (D). Succinate, a metabolite predicted to be substantially utilized by C. difficile in the CR-comm model (FIG. 3 (A), is predicted to have no effect on C. difficile growth in GB model (FIG. 3 (E)). The inconsistencies between the inferred interactions highlight the different modalities captured by each model. In sum, the inferred interactions in the consumer resource and ML models provides deeper insights into the contributions of resource competition versus other classes of interactions that may dominate C. difficile abundance in communities.

Model-Optimized Communities Inhibit C. difficile Across Different Nutrient Environments

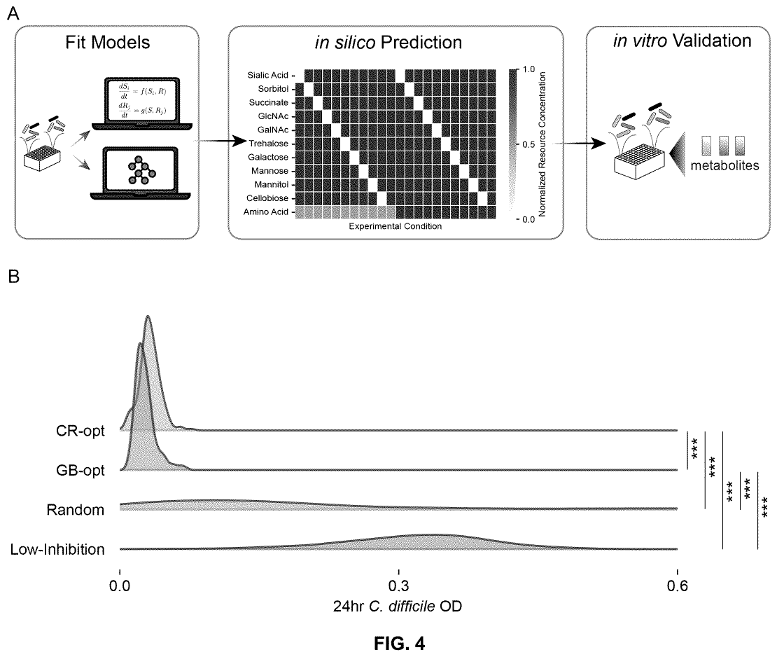

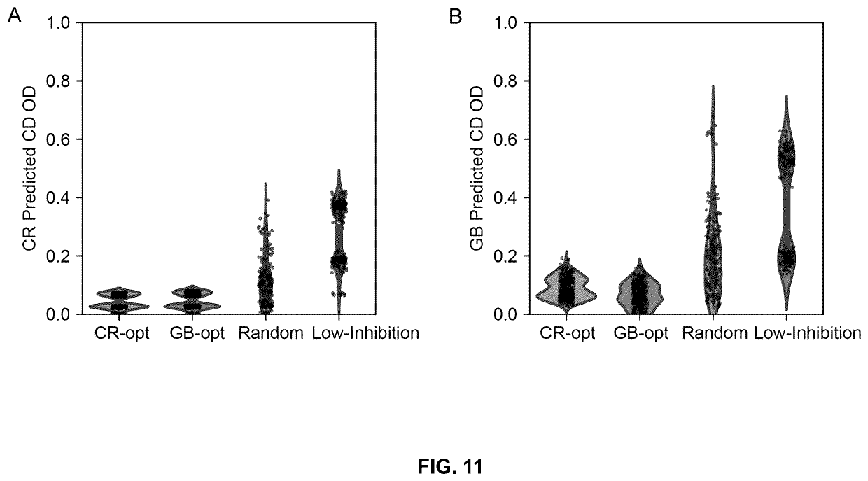

Given the fluctuating and unpredictable nutrient conditions in the mammalian gut^{25,26}, a key question is determining which microbial communities can consistently suppress C. difficile across distinct nutrient environments. The CR-comm and GB models fit to all data were separately used to predict the abundance of C. difficile at 24 hours in all four-member communities in 22 nutrient environments (FIGS. 4 (A) and 11). These conditions included the presence of all carbohydrates (all 10 added at 1 g/L, total 10 g/L) and leave-one-carbohydrate-out media (all but one added at 1 g/L, total 9 g/L) at two different amino acid levels (5 g/L and 10 g/L). To test the model predictions, we selected 15 communities with the lowest predicted maximum C. difficile abundance at 24 hours across all in silico media conditions for experimental characterization in 22 media conditions (CR-opt and GB-opt for communities optimized using the CR-comm model and GB model, respectively). As a control, we selected 15 random communities (Random communities) to evaluate the ability of the models to identify communities that inhibit C. difficile better than random. Finally, we selected 15 communities predicted by both CR-comm and GB models to be among the 250 communities with the highest minimum C. difficile abundance (Low-Inhibition communities) to evaluate the ability of the models to distinguish inhibitory versus non-inhibitory C. difficile communities.

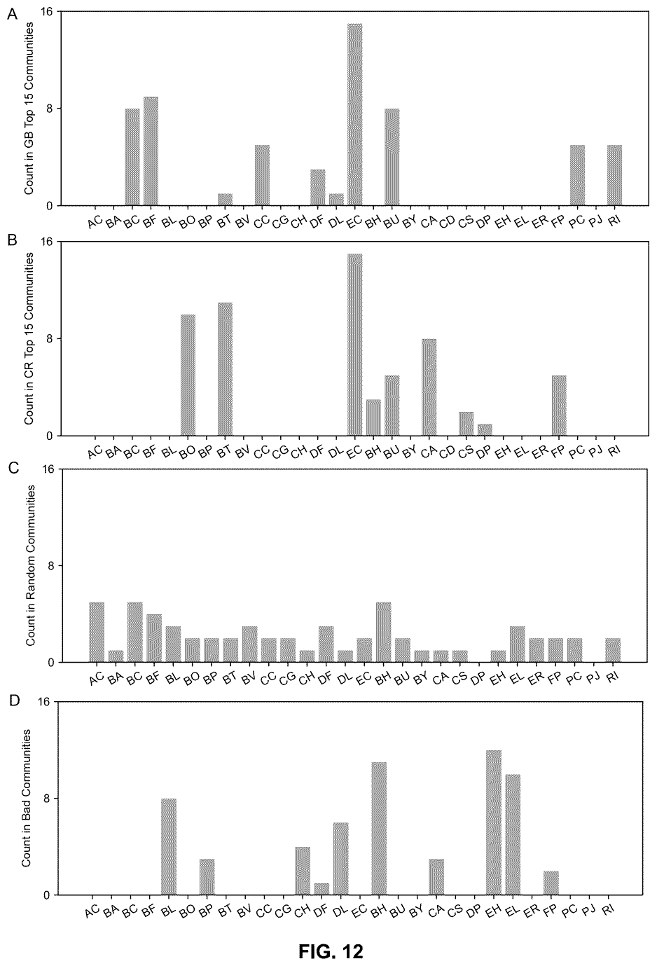

Due to different inductive biases of the consumer resource and machine learning model, the species compositions of CR-opt and GB-opt can provide insights into different solutions for anti-C. difficile communities. EC is present in all model-optimized communities (FIG. 12 (A, B), consistent with its negative impact on C. difficile based on SHAP analysis using the GB model (FIG. 3 (D)) and the inferred high metabolic niche overlap in the CR-comm model (FIG. 10 (B)). While EC was a universal feature, BC, BF, and BU frequently occur in CR-opt (FIG. 12 (A)), whereas BO, BT, and CA are enriched in GB-opt (FIG. 12 (B). As expected, the Random communities consist of a uniform sampling of the species in the design space (FIG. 12 (C). The Low-Inhibition communities are enriched with BL, BH, EH, and EL (FIG. 12 (D), indicating that these species do not inhibit the growth of C. difficile. Consistent with this result, EH, BL, and BH are predicted to have low metabolic niche overlap with C. difficile in the CR-comm model (FIG. 10 (B)) and positively impact C. difficile based on the SHAP analysis using the GB model (EL, EH and BH are within the top 4 highest values) (FIG. 3 (D)).

To evaluate the predictive capability of the models, we experimentally characterized all communities by assembling the communities in 22 media conditions tested in silico and measured community composition based on 16S rRNA gene sequencing and total community biomass based on OD600. The absolute abundance of C. difficile was low in CR-opt and GB-opt communities compared to Random and Low-Inhibition communities (mean OD600 for C. difficile was 0.03 and 0.02 for CR-opt and GB-opt, respectively) across all media conditions (FIG. 4 (B)), consistent with the model predictions (FIG. 11). Notably, the GB-opt communities displayed significantly lower C. difficile abundance than CR-opt (FIG. 4 (B)). This implies that the gradient boosting model effectively captures critical inhibitory interactions influencing C. difficile that the consumer-resource fails to capture. The Random communities displayed a wide range of C. difficile abundance and significantly higher mean (mean OD600 of 0.18) than CR-opt and GB-opt (FIG. 4 (B)). This highlights that the model-designed communities displayed significantly improved C. difficile inhibition performance than randomly selected communities of the same richness. Finally, the Low-Inhibition communities displayed the highest 24-hour C. difficile abundance with a mean OD600 of 0.32 (FIG. 4 (B)), consistent with the model predictions (FIG. 11).

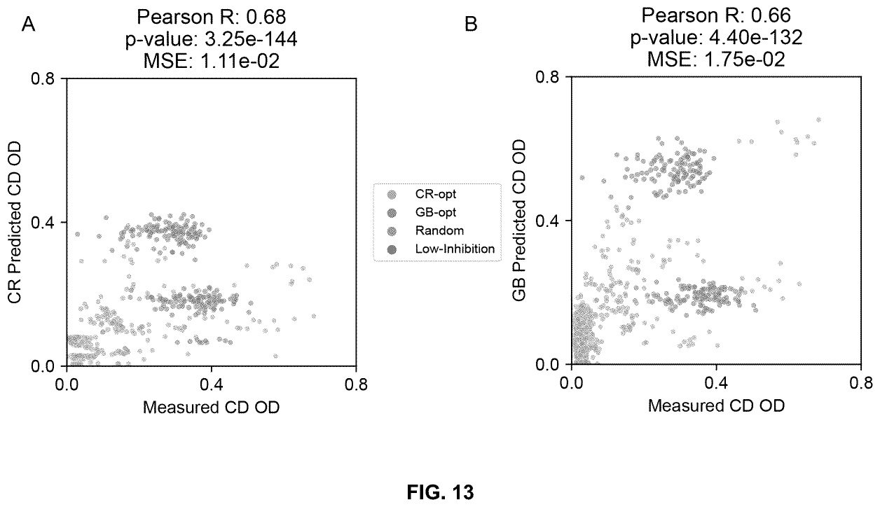

Based on their initial metabolite concentration, species compositions, and phylogenetic relatedness, the GB-opt, CR-opt, Low-Inhibition and Random conditions were spread broadly across the design space (FIG. 2 (D)). Therefore, there are multiple distinct solutions for C. difficile inhibition within the design space. To evaluate model prediction performance on these data, the models were used to predict C. difficile abundance in the validation dataset. Although the GB-opt and CR-opt communities were designed to target low C. difficile abundance, the GB and CR-comm models displayed good prediction performance across all data in the validation set (Pearson correlation of 0.68 with p-value of 3.25*10⁻¹⁴⁴ for the CR-comm model, whereas GB model predictions have Pearson correlation of 0.66 with p-value of 4.40*10⁻¹³²) (FIG. 13). In sum, both models successfully designed communities to significantly inhibit C. difficile abundance in vitro compared to randomly selected communities or communities that were predicted to maximize C. difficile abundance.

Metabolic Profiles Provide Insights into Mechanisms of Resource Competition

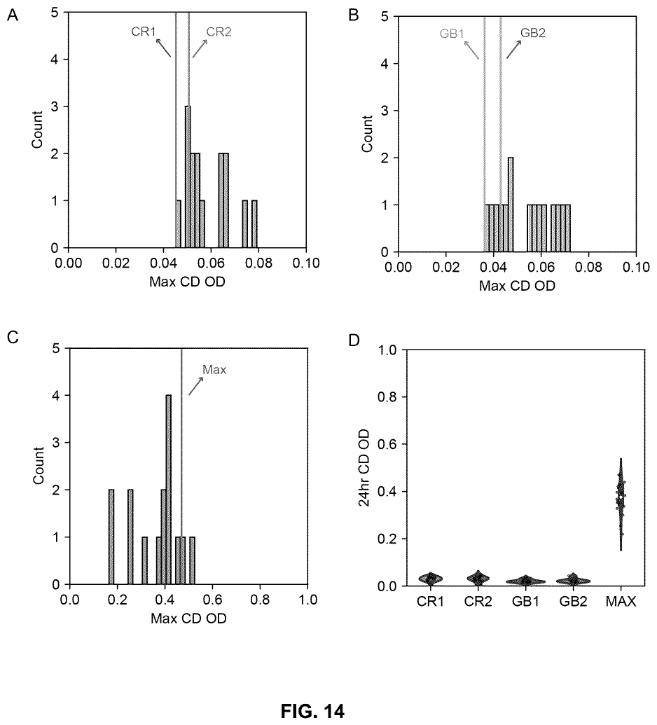

To reveal the changes in key metabolites in model-designed communities, we used exo-metabolomic profiling on monoculture and community supernatants. We selected four communities that displayed low C. difficile abundance across 22 media conditions (FIG. 14 (A, B, D). From CR-opt we selected CR1 (BO, BH, EC, and CA) and CR2 (BO, BT, EC, and FP) and from GB-opt we selected GB1 (BF, BU, EC, and RI) and GB2 (BC, CC, EC, and DF).

We performed exo-metabolomic profiling on the supernatant of C. difficile monoculture, five model-designed communities, and sixteen leave-one-species-out sub-communities cultured in media with all carbohydrates (each carbohydrate at 1 g/L) and 10 g/L amino acids. We determined the absolute metabolite concentration via targeted metabolomics on all carbohydrates in the design space and amino acids utilized in Stickland metabolism (proline and leucine) 51,59,60. We computed the change in concentration of a given metabolite in each culture between hour 0 and hour 24, minus the change in the given metabolite in the blank media between hour 0 and hour 24 (Methods). Peaks for GalNAc/GlcNAc, sorbitol/mannitol, galactose/mannose or cellobiose/trehalose could not be resolved by UHPLC-MS and thus are reported as pairs of metabolites.

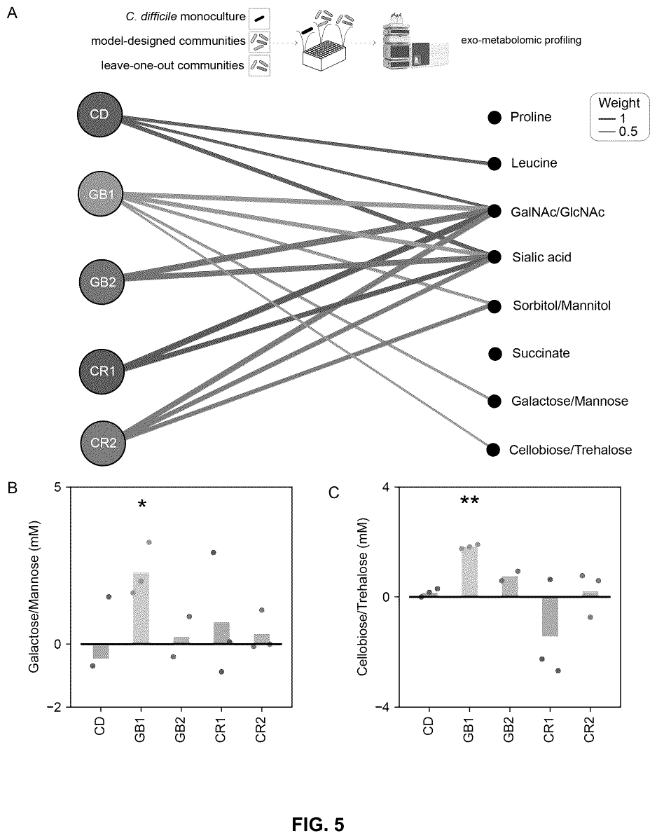

Our results demonstrate that C. difficile monoculture displayed significant utilization of leucine, GalNAc/GlcNAc, and sialic acid (FIG. 5 (A)), highlighting the metabolic niche of C. difficile in media containing all carbohydrates. While Cᵢⱼ from CR-mono model predicted utilization of amino acids and GlcNAc, the CR-comm model failed to capture C. difficile utilization of sialic acid (FIG. 7 (C)). All model-optimized communities exhibited significant utilization of GalNAc/GlcNAc and sialic acid (FIG. 5 (A)). Since these communities robustly inhibited C. difficile (FIG. 14 (D)), GalNAc, GlcNac, and sialic acid may mediate resource competition with C. difficile and thus contribute to the observed C. difficile growth inhibition. The communities GB1 and CR2 utilized sorbitol/mannitol, whereas GB1 also uniquely depleted galactose/mannose and cellobiose/trehalose (FIG. 5 (B, C). Notably, GB1 displayed the broadest range of resource utilization of the measured metabolites compared to the other model-designed communities (FIG. 5 (A)).

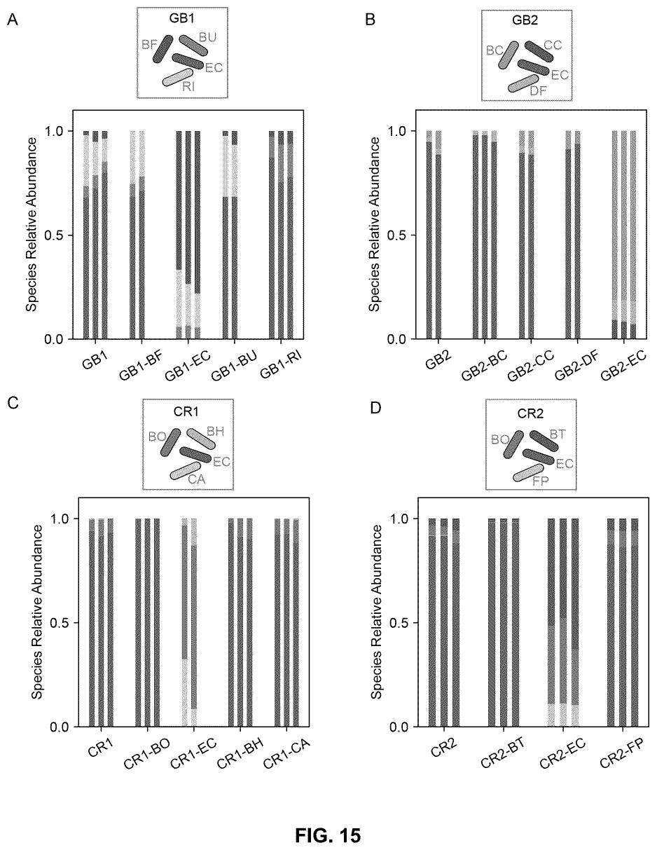

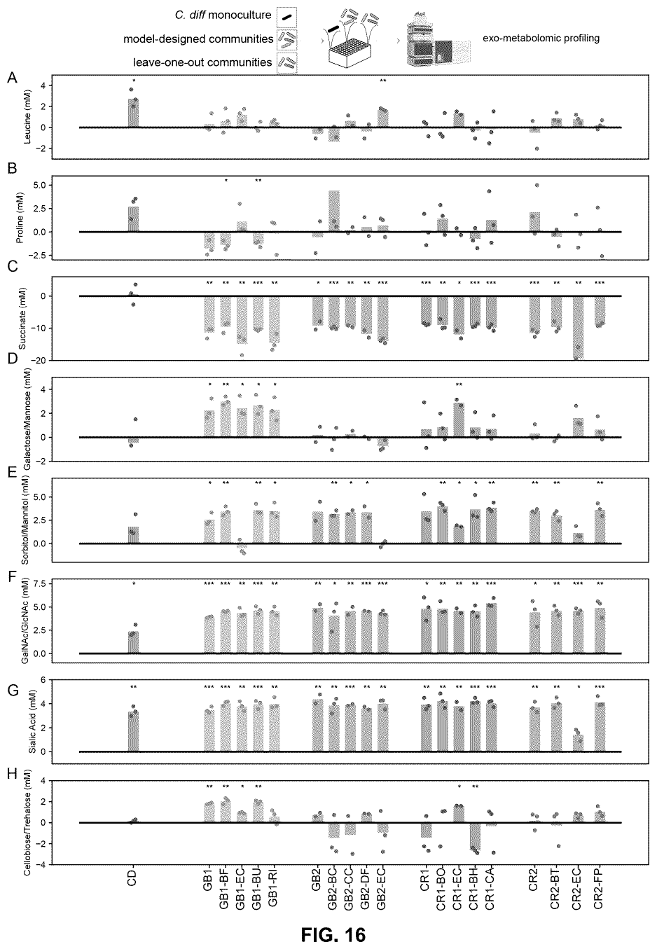

Exo-metabolomic characterization of single species dropout communities can provide deeper insights into the contributions of individual species on resource utilization in the context of the community (FIG. 15). The full communities displayed differences in metabolite profiles compared to their respective sub-communities (FIG. 16). For example, removal of EC significantly increased the consumption of leucine in GB2 (FIG. 16 (A), galactose/mannose in CR1 (FIG. 16 (D)), and cellobiose/trehalose in CR1 (FIG. 16 (H)). This implies that EC inhibits the utilization of these metabolites by competitive exclusion of specific species, consistent with an enhancement in the relative abundance of BC in GB2 and BO in CR1 in the absence of EC (FIG. 15). By contrast, elimination of EC significantly reduced the consumption of sorbitol/mannitol in GB1, GB2, CR2 (FIG. 16 (E)) and cellobiose/trehalose in GB1 (FIG. 16 (H)), suggesting EC utilized these metabolites. All communities and subcommunities displayed significant utilization of GalNAc/GlcNac (FIG. 16 (F) and sialic acid (FIG. 16 (G), highlighting a common resource utilization pattern of the communities that was robust to removal of individual species. This is consistent with the inferred Cᵢⱼ parameters in the CR-comm model indicating that each model-optimized community exhibits functional redundancy in the utilization of GalNAc, GlcNAc, and sialic acid (EC, BF, BU, BC, BO, and BT) (FIG. 3 (A).

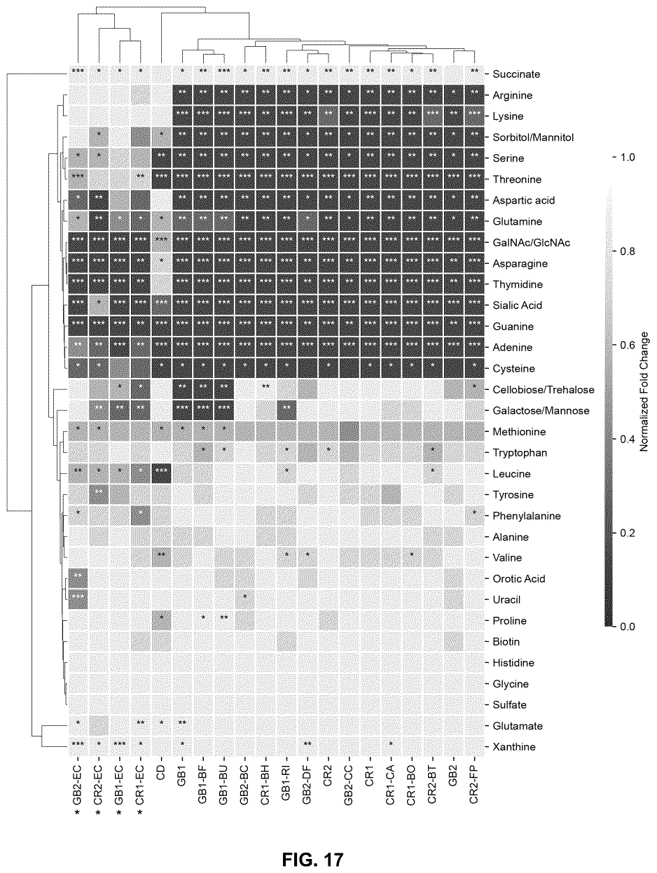

Beyond the key resources, we evaluated the fold changes of other metabolites in the media due to the lack of absolute abundance quantification. C. difficile monoculture exhibited significant consumption of serine, threonine, glutamine, guanine, adenine, cysteine, methionine, and valine (FIG. 17), consistent with previous reports of amino acid and purine nucleotide utilization by C. difficile⁶¹⁻⁶³. All communities containing EC have similar resource utilization landscape (FIG. 17), indicating that EC had a major role in defining the resource utilization profile of the communities. Subcommunities and communities with EC display significant depletion of arginine and lysine, suggesting EC was critical for the consumption of these two metabolites. Of all communities, the metabolite profile of GB1 was the most similar to C. difficile monoculture based on a hierarchical clustering (FIG. 17), suggesting GB1 had similar metabolite release/utilization profiles to C. difficile. In sum, model-optimized communities can consume metabolites utilized by C. difficile, and GB1 displayed a unique resource utilizations pattern than the other model-designed communities.

### In Vitro Summary

The species composition of each of the 30 communities tested in vitro and validated to be inhibitory to C. difficile (FIG. 4B) is shown in Table 1.

TABLE 1
Communities tested in vitro and validated to be inhibitory to C. difficile.

| Community | EC | BU | BT | BO | BC | BF | CA | CC | FP | PC | RI | DF | BH | CS | DL | DP |
|---|---|---|---|---|---|---|---|---|---|---|---|---|---|---|---|---|
| EC, BU, BT, BO | 1 | 1 | 1 | 1 | 0 | 0 | 0 | 0 | 0 | 0 | 0 | 0 | 0 | 0 | 0 | 0 |
| EC, BU, BT, CA | 1 | 1 | 1 | 0 | 0 | 0 | 1 | 0 | 0 | 0 | 0 | 0 | 0 | 0 | 0 | 0 |
| EC, BU, BT, FP | 1 | 1 | 1 | 0 | 0 | 0 | 0 | 0 | 1 | 0 | 0 | 0 | 0 | 0 | 0 | 0 |
| EC, BU, BO, CA | 1 | 1 | 0 | 1 | 0 | 0 | 1 | 0 | 0 | 0 | 0 | 0 | 0 | 0 | 0 | 0 |
| EC, BU, BO, FP | 1 | 1 | 0 | 1 | 0 | 0 | 0 | 0 | 1 | 0 | 0 | 0 | 0 | 0 | 0 | 0 |
| EC, BU, BC, BF | 1 | 1 | 0 | 0 | 1 | 1 | 0 | 0 | 0 | 0 | 0 | 0 | 0 | 0 | 0 | 0 |
| EC, BU, BC, RI | 1 | 1 | 0 | 0 | 1 | 0 | 0 | 0 | 0 | 0 | 1 | 0 | 0 | 0 | 0 | 0 |
| EC, BU, BF, CC | 1 | 1 | 0 | 0 | 0 | 1 | 0 | 1 | 0 | 0 | 0 | 0 | 0 | 0 | 0 | 0 |
| EC, BU, BF, PC | 1 | 1 | 0 | 0 | 0 | 1 | 0 | 0 | 0 | 1 | 0 | 0 | 0 | 0 | 0 | 0 |
| EC, BU, BF, RI | 1 | 1 | 0 | 0 | 0 | 1 | 0 | 0 | 0 | 0 | 1 | 0 | 0 | 0 | 0 | 0 |
| EC, BU, BF, DF | 1 | 1 | 0 | 0 | 0 | 1 | 0 | 0 | 0 | 0 | 0 | 1 | 0 | 0 | 0 | 0 |
| EC, BU, PC, RI | 1 | 1 | 0 | 0 | 0 | 0 | 0 | 0 | 0 | 1 | 1 | 0 | 0 | 0 | 0 | 0 |
| EC, BU, RI, DL | 1 | 1 | 0 | 0 | 0 | 0 | 0 | 0 | 0 | 0 | 1 | 0 | 0 | 0 | 1 | 0 |
| EC, BT, BO, CA | 1 | 0 | 1 | 1 | 0 | 0 | 1 | 0 | 0 | 0 | 0 | 0 | 0 | 0 | 0 | 0 |
| EC, BT, BO, FP | 1 | 0 | 1 | 1 | 0 | 0 | 0 | 0 | 1 | 0 | 0 | 0 | 0 | 0 | 0 | 0 |
| EC, BT, BO, BH | 1 | 0 | 1 | 1 | 0 | 0 | 0 | 0 | 0 | 0 | 0 | 0 | 1 | 0 | 0 | 0 |
| EC, BT, BO, CA | 1 | 0 | 1 | 1 | 0 | 0 | 0 | 0 | 0 | 0 | 0 | 0 | 0 | 1 | 0 | 0 |
| EC, BT, BO, DP | 1 | 0 | 1 | 1 | 0 | 0 | 0 | 0 | 0 | 0 | 0 | 0 | 0 | 0 | 0 | 1 |
| EC, BT, BF, CC | 1 | 0 | 1 | 0 | 0 | 1 | 0 | 1 | 0 | 0 | 0 | 0 | 0 | 0 | 0 | 0 |
| EC, BT, CA, FP | 1 | 0 | 1 | 0 | 0 | 0 | 1 | 0 | 1 | 0 | 0 | 0 | 0 | 0 | 0 | 0 |
| EC, BT, CA, BH | 1 | 0 | 1 | 0 | 0 | 0 | 1 | 0 | 0 | 0 | 0 | 0 | 1 | 0 | 0 | 0 |
| EC, BT, CA, CS | 1 | 0 | 1 | 0 | 0 | 0 | 1 | 0 | 0 | 0 | 0 | 0 | 0 | 1 | 0 | 0 |
| EC, BO, CA, FP | 1 | 0 | 0 | 1 | 0 | 0 | 1 | 0 | 1 | 0 | 0 | 0 | 0 | 0 | 0 | 0 |
| EC, BO, CA, BH | 1 | 0 | 0 | 1 | 0 | 0 | 1 | 0 | 0 | 0 | 0 | 0 | 1 | 0 | 0 | 0 |
| EC, BC, BF, CC | 1 | 0 | 0 | 0 | 1 | 1 | 0 | 1 | 0 | 0 | 0 | 0 | 0 | 0 | 0 | 0 |
| EC, BC, BF, PC | 1 | 0 | 0 | 0 | 1 | 1 | 0 | 0 | 0 | 1 | 0 | 0 | 0 | 0 | 0 | 0 |
| EC, BC, BF, DF | 1 | 0 | 0 | 0 | 1 | 1 | 0 | 0 | 0 | 0 | 0 | 1 | 0 | 0 | 0 | 0 |
| EC, BC, CC, PC | 1 | 0 | 0 | 0 | 1 | 0 | 0 | 1 | 0 | 1 | 0 | 0 | 0 | 0 | 0 | 0 |
| EC, BC, CC, DF | 1 | 0 | 0 | 0 | 1 | 0 | 0 | 1 | 0 | 0 | 0 | 1 | 0 | 0 | 0 | 0 |
| EC, BC, PC, RI | 1 | 0 | 0 | 0 | 1 | 0 | 0 | 0 | 0 | 1 | 1 | 0 | 0 | 0 | 0 | 0 |

Each row is a distinct community and species presence/absence is indicated by 1/0.
See FIG. 1 for definitions of the species abbreviations.

Optimized Communities Confer Distinct Effects on C. difficile in the Mammalian Gut

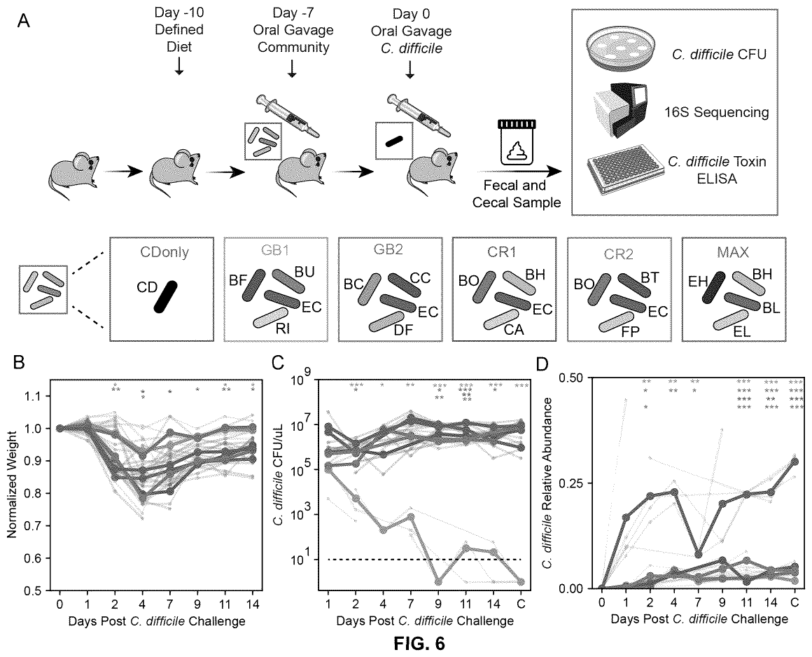

To evaluate the effect of the model-designed communities on C. difficile in the mammalian gut, we used a germ-free mouse model orally gavaged with each community or no community as a control. In addition to the four model-optimized communities characterized by exo-metabolomics, we selected a representative community that displayed a high C. difficile absolute abundance (24 hr) across media conditions from the Weak group referred to as Max (FIG. 14 (C, D). Germ-free mice were partitioned into six groups, including GB1, GB2, CR1, CR2, Max, and a no community group (C. difficile alone or Cdonly) (FIG. 6 (A)). Female mice (8-12 weeks old) were fed a standard grain-based chow diet (Envigo 2018S, containing 18.4% protein, 6.0% fat, 44.2% carbohydrate, and 18.5% total fiber) for three days before oral gavage of communities in 5 of 6 conditions (Cdonly group had no community gavage) (FIG. 6 (A)). One week after oral gavage with the communities (or no community), all groups of mice were orally gavaged with the same number of C. difficile vegetative cells. We used vegetative cells as opposed to spores for consistency with our in vitro experimental design and computational model training (FIG. 6 (A)) and because of their in vivo use in other studies⁶⁴⁻⁶⁶ To evaluate the effects of each treatment on the host, we weighed each mouse every 2-3 days and collected fecal samples for determining the colony forming unit (CFU) of C. difficile and species relative abundance based on 16S rRNA gene sequencing (FIG. 6 (A)). Following two weeks of C. difficile challenge, mice were sacrificed and cecal contents were analyzed for C. difficile colony forming units (CFU), species relative abundance, and C. difficile toxin tcdA and tcdB concentration using an Enzyme-linked immunosorbent assay (ELISA) (FIG. 6 (A)).

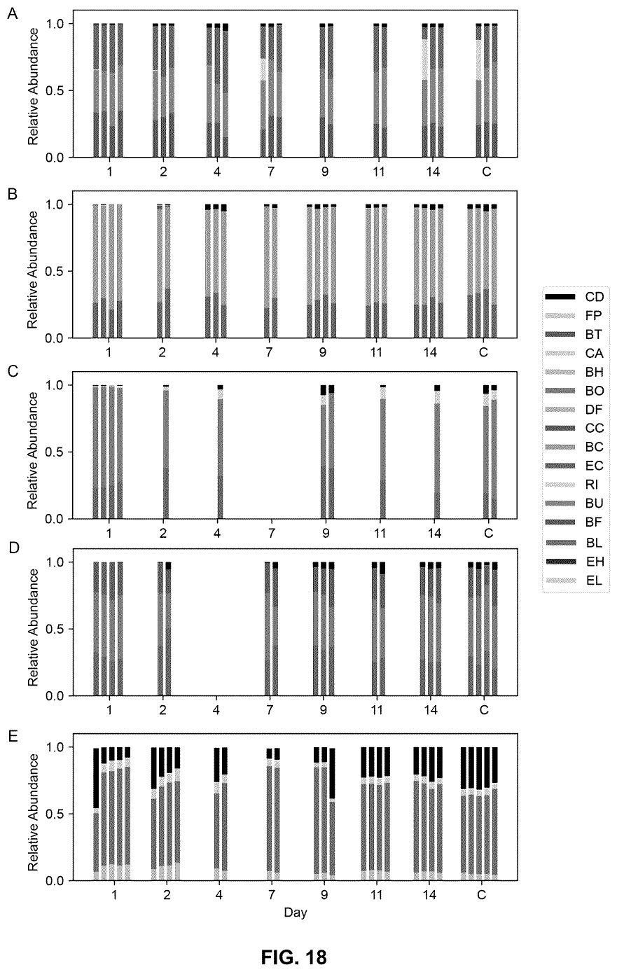

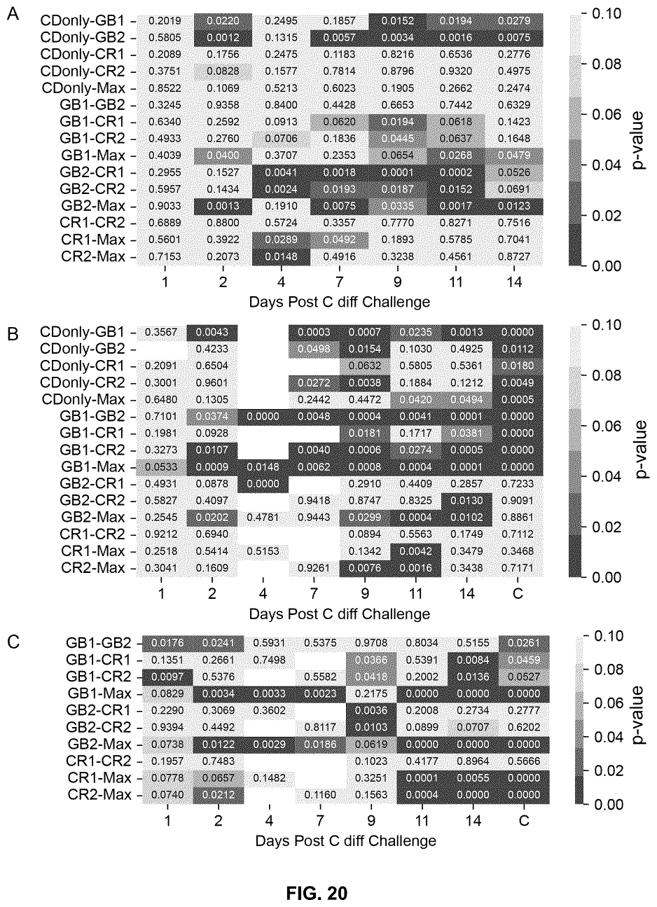

The comparison of each model-optimized community to the Max can reveal the effects of communities designed for C. difficile inhibition while controlling for the physiological impact of colonization of germ-free mice by commensal bacteria more generally. The normalized weight of mice decreased two days after C. difficile challenge for all groups (FIGS. 6 (B) and 20 (A)). The GB1 and GB2 groups displayed significantly higher normalized weight than the Max group on days 2, 11, and 14 following C. difficile challenge as well as significantly higher normalized weight than Cdonly group on days 2, 9, 11, and 14. Therefore, communities designed by the gradient boosting model to inhibit C. difficile ameliorated the severity of infection compared to the controls. By contrast, CR1 and CR2 displayed significantly lower normalized weight than the Max group on day 4 and no significant difference compared to the Cdonly group on any day, indicating that these communities did not ameliorate disease severity compared to the controls. For all model-designed community groups except GB1, the absolute abundance of C. difficile was maintained at 10⁵-10⁷ CFU/UI (FIGS. 6 (C) and 20 (B)), indicating no significant difference in C. difficile colonization among these groups. Notably, for GB1, C. difficile absolute abundance decreased and was undetectable at day 9, demonstrating that this community substantially inhibited C. difficile colonization of the murine gut. While C. difficile absolute abundance has been used to evaluate efficacy for therapeutic applications, C. difficile relative abundance provide additional insights into colonization in the context of the community. All model-optimized communities displayed significantly lower C. difficile relative abundance on days 11 and 14 and in cecal contents than the Max group (FIGS. 6 (D), 18, and 20 (C)).

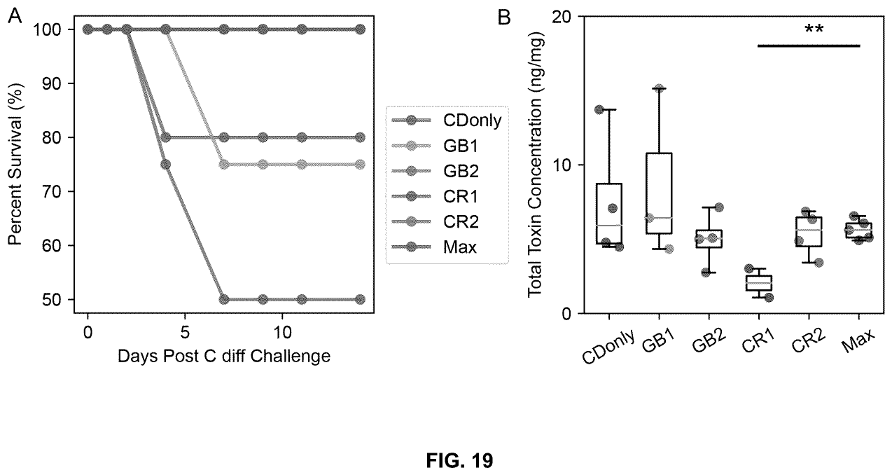

The mapping between the degree of colonization of C. difficile and the response of the host was more complex. CR1 displayed 50% survival, GB1 had 75% survival, and Cdonly had 80% survival whereas the other groups displayed 100% survival (FIG. 19 (A)), suggesting variable survival among the groups for this sample size. The total toxin concentration analysis based on ELISA was similar across groups with a significant difference between CR1 and Max (FIG. 19 (B)). Since ELISA quantifies total toxin concentration rather than activity, the toxin activity may trend better with C. difficile abundance. In sum, our results feature GB1 as substantially ameliorating disease severity based on trends in weight over time and reducing C. difficile abundance to below the detection limit in the mammalian gut.

### Discussion

Recent clinical advancements demonstrate the tremendous potential of using defined consortia for treating C. difficile infection. For example, VE303 (Vedanta Biosciences) is undergoing a Phase 2 clinical trial⁸. However, we lack an understanding of the mechanisms of action that are most effective against C. difficile and the potential failure modes. Resource competition was previously highlighted as an influential mechanism in C. difficile inhibition using defined consortia in mouse models^{21,67}. Leveraging high-throughput bottom-up microbial community assembly, Bayesian experimental design, and machine learning, we performed model-guided exploration across a vast metabolite-species design space to discover C. difficile-inhibitory communities. To provide key insights into the role of resource competition in C. difficile inhibition, we used a mechanistic and a machine learning model to capture the patterns in the data and design new communities for optimized C. difficile inhibition across different nutrient environments.

Computational models display different inductive biases⁶⁸ and thus can identify distinct alternative solutions. Therefore, leveraging different computational models for microbiome engineering could reveal distinct classes of mechanisms and solutions for deploying in environments with uncertain environmental conditions. The GB1 community designed by the machine learning model displays a unique resource utilization profile, reduces weight loss in a C. difficile infection model, and substantially reduces C. difficile from cecal content over a two-week period to below the detection limit. Previous studies have demonstrated reduced C. difficile colonization in a murine model by defined consortia^{21,47} and clearance of C. difficile in a murine model by the resident microbiome⁶⁹. To our knowledge, this is the first study to report clearance of C. difficile by a defined community to an undetectable level.

Comparing the prediction and forward design performance of the consumer resource and machine learning can shed light into the contribution of resource competition on C. difficile inhibition. Both models identified communities that significantly inhibit C. difficile growth compared to randomly selected communities and low-inhibition communities across in vitro media conditions (FIG. 4 (B)). This suggests that communities designed based on resource competition displayed similar inhibition compared to communities designed based on all interaction modalities. Therefore, resource competition was a major mechanism shaping C. difficile growth in vitro. On the other hand, consumer resource model designed communities CR1 and CR2 did not ameliorate disease severity based on the trends in weight over time (FIG. 6 (B)) and did not limit C. difficile colonization in fecal and cecal samples (FIG. 6 (C)). By contrast, GB1 and GB2 significantly improves disease severity (FIG. 6 (B) and GB1 reduces C. difficile colonization from the murine gut to undetectable levels (FIG. 6 (C)). Therefore, with the limited number of communities tested in mice, the machine learning model was superior to the consumer resource model in identifying communities that can inhibit C. difficile in the murine gut. A possible explanation is that the machine learning model captures influential and complex interactions that impact C. difficile colonization of the mammalian gut.

All communities and subcommunities displayed significant utilization of GalNAc/GlcNac (FIG. 16 (F)) and sialic acid (FIG. 16 (G)), highlighting robust utilization of these metabolites by different species, in accordance with the inferred Cᵢⱼ parameters of the CR-comm model (FIG. 3 (A)). However, the consumer resource model adopted in this study can be modified to capture additional key resources mediating interactions with C. difficile. For example, instead of modeling the pool of amino acids, individual amino acids fueling C. difficile Stickland metabolism (e.g. leucine and proline) 51,59,60 can be represented to better resolve communities that compete with C. difficile over these key nutrients. Further, the discovery of additional resources used by C. difficile in the murine gut could be incorporated into the high-throughput in vitro experimental workflows. Consistent with previous reports of amino acid and purine nucleotide utilization by C. difficile⁶¹⁻⁶³, we observed serine, threonine, glutamine, guanine, adenine, cysteine, methionine, and valine utilization by C. difficile monoculture (FIG. 17), potentially representing novel targets of resource competition for C. difficile inhibition. Finally, while we did not perform high-throughput measurements of metabolite concentrations, high-throughput metabolite measurement methods^{70,71} could be incorporated into future DTL cycles to inform the computational models and thus potentially improve model performance.

The inferred parameters of the consumer resource model and SHAP analysis of the machine learning model uncovers interactions that contribute to C. difficile inhibition across the species-resource design space. For instance, EC, BC, and BT are strong C. difficile inhibitors in both models (FIG. 3 (A, D)) and BH and EH are consistently poor C. difficile inhibitors (FIG. 3 (A, D). While the consumer resource model predicts strong utilization of succinate by C. difficile, the gradient boosting model displays a negligible interaction between succinate and C. difficile (FIG. 3 (A, E)). In SHAP analysis, collinearity among variables can make it difficult to accurately determine each variable's unique contribution. For example, SHAP analysis may negligibly effect from succinate on the prediction of C. difficile abundance due to the balance between the growth promotion from succinate and the competition with succinate-utilizing species that inhibit C. difficile^{72,73}.

The relationship between the designed communities, C. difficile colonization and the host response are complex in the murine model. Although C. difficile was cleared from the cecum to below the detection limit (FIG. 6 (C)), the GB1 group had the second lowest survival rate (FIG. 19 (A)) and its cecal content toxin concentrations via ELISA were similar to the Cdonly and Max groups (FIG. 19 (B)). These discrepancies could be contributed to low statistical power in the mouse experimental design, low specificity of the ELISA assay, and the complexities related to C. difficile toxin production and immunological stability. An avenue of future research is to increase the number of biological replicates in each group to obtain a more robust assessment of survival across treatments^{74,75}. The ELISA assay quantifies the amount of toxin protein in sample, rather than the activity of toxins^{76,77}, which could lead to discrepancy between toxin load and disease outcome. Activity assays like Vero cell assays could be used instead to directly measure the activity of C. difficile toxins^{78,79}. Certain metabolite in gut, such as bile acids, may bind to and inactivate C. difficile toxin⁸⁰, contributing to the difference between C. difficile toxin amount and activity. Additionally, toxins may persist in stool due to mice coprophagy⁸¹. Therefore, C. difficile CFU and toxin concentration may correlate better in earlier time points, as previously shown at 24 hours after C. difficile challenge⁴⁷. Finally, while we studied vegetative C. difficile cells in this study, we could incorporate sporulation and germination82 into the computational model to better capture C. difficile life cycles during infection.

A major goal for microbiome engineering is to discover generalizable design principles for robust pathogen inhibition in response to environmental variability. Our model-guided community design approach using a mechanistic model and a machine learning model, coupled with Bayesian experimental design, establishes a foundation for the efficient and rational design of functional microbial communities across immense species-metabolite design spaces. The consistency of C. difficile inhibition in our in vitro system and murine model demonstrates the potential translatability of certain mechanisms of inhibition. Our framework of model-guided community design can be applied to other pathogens as well as other mechanisms of interaction to facilitate the rational design of highly functional and robust microbial therapeutics.

### Material and Methods

### Strain Maintenance and Culturing

All anaerobic culturing was carried out in anaerobic chambers with an atmosphere of 2±0.5% H₂, 15±1% CO₂ and balance N₂. All prepared media and materials for anaerobic experiments were placed in the chamber at least overnight before use to equilibrate with the chamber atmosphere. The permanent stocks of each strain used in these examples were stored in 25% glycerol at −80 °C. Batches of single-use glycerol stocks were produced for each strain by first growing a culture from the permanent stock in ABB media to stationary phase, mixing the culture in an equal volume of 50% glycerol, and aliquoting 400 mL into Matrix Tubes (ThermoFisher) for storage at −80 °C Quality control for each batch of single-use glycerol stocks included Illumina sequencing of 16S rDNA isolated from pellets of the aliquoted mixture to verify the identity of the organism. For each experiment, precultures of each species were prepared by thawing a single-use glycerol stock (SUGS) and adding 100 ml of SUGS to 1 mL fresh ABB media for incubation at 37 °C for 24 hours or 48 hours.

### Monoculture and Community Culturing Dynamic Growth Quantification

Each species' preculture was diluted to an OD600 of 0.01 (Tecan F200 Plate Reader) in a defined media²⁴ designed in a previous study. Media Ph is titrated to 7 using sodium hydroxide and hydrochloric acid, based on measurements obtained from a Ph probe (Mettler-Toledo). Precultures were then aliquoted into three replicates of 1.5 mL each in a 96 Deep Well (96DW) plate and covered with a semi-permeable membrane (Diversified Biotech) and incubated at 37 °C without shaking. At each time point, samples were mixed by pipetting up and down 5 times before transferring 200 UI to 96 well microplate for measurement of OD by the plate reader (Tecan F200 Plate Reader). Cell pellets were spun down and saved at −80 °C at each timepoint for species abundance quantification.

Genomic DNA Extraction from Cell Pellets

Genomic DNA was extracted from cell pellets using a modified version of the Qiagen Dneasy Blood and Tissue Kit protocol. First, pellets in 96DW plates were removed from −80 °C and thawed in a room temperature water bath. Each pellet was resuspended in 180 ml of enzymatic lysis buffer (20 mM Tris-HCl (Invitrogen), 2 mM sodium EDTA (Sigma-Aldrich), 1.2% Triton X-100 (Sigma-Aldrich), 20 mg/ml lysozyme from chicken egg white (Sigma-Aldrich)). Plates were then covered with a foil seal and incubated at 37 °C for 30 min with orbital shaking at 600 RPM. Then, 25 mL of 20 mg mL⁻¹ Proteinase K (VWR) and 200 ml of Buffer AL (QIAGEN) were added to each sample before mixing with a pipette. Plates were then covered by a foil seal and incubated at 56 °C for 30 min with orbital shaking at 600 RPM. Next, 200 mL of 100% ethanol (Koptec) was added to each sample before mixing and samples were transferred to a nucleic acid binding (NAB) plate (Pall) on a vacuum manifold with a 96DW collection plate. Each well in the NAB plate was then washed once with 500 mL buffer AW1 (QIAGEN) and once with 500 ml of buffer AW2 (QIAGEN). Samples were then eluted into a clean 96DW plate from each well using 110 mL of buffer AE (QIAGEN) preheated to 56 °C Genomic DNA samples were stored at −20 °C until further processing.

### Sequencing Library Preparation

Genomic DNA concentrations were measured using a SYBR Green fluorescence assay and then normalized to a concentration of 1 ng mL⁻¹ by diluting in molecular grade water using a Tecan Evo Liquid Handling Robot. First, genomic DNA samples were removed from −20 °C and thawed in a room temperature water bath. Then, 1 ml of each sample was combined with 95 mL of SYBR Green (Invitrogen) diluted by a factor of 100 in TE buffer (Integrated DNA Technologies) in a black 384-well microplate. This process was repeated with two replicates of each DNA standard with concentrations of 0, 0.5, 1, 2, 4, and 6 ng mL⁻¹. Each sample was then measured for fluorescence with an excitation/emission of 485/535 nm using a Tecan Spark plate reader. Concentrations of each sample were calculated using the standard curve and a custom Python script was used to compute the dilution factors and write a worklist for the Tecan Evo Liquid Handling Robot to normalize each sample to 1 ng mL⁻¹ in molecular grade water. Samples with DNA concentration <1 ng mL⁻¹ were not diluted. Diluted genomic DNA samples were stored at −20 °C until further processing.

Amplicon libraries were generated from diluted genomic DNA samples by PCR amplification of the V3-V4 of the 16S rRNA gene using custom dual-indexed primers for multiplexed next-generation amplicon sequencing on Illumina platforms (Method adapted from Venturelli et al. Mol. Syst. Biol., 2018). Primers were arrayed in skirted 96-well PCR plates (VWR) using an acoustic liquid handling robot (Labcyte Echo 550) such that each well received a different combination of one forward and one reverse primer (0.1 mL of each). After liquid evaporated, dry primers were stored at −20 °C. Primers were resuspended in 15 mL PCR master mix (0.2 mL Phusion High Fidelity DNA Polymerase (Thermo Scientific), 0.4 mL 10 mM Dntp solution (New England Biolabs), 4 mL 5×47lostri HF buffer (Thermo Scientific), 4 mL 5 M Betaine (Sigma-Aldrich), 6.4 mL Water) and 5 mL of normalized genomic DNA to give a final concentration of 0.05 mM of each primer. Primer plates were sealed with Microplate B seals (Bio-Rad) and PCR was performed using a Bio-Rad C1000 Thermal Cycler with the following program: initial denaturation at 98 °C (30 s); 25 cycles of denaturation at 98 °C (10 s), annealing at 60 °C (30 s), extension at 72 °C (60 s); and final extension at 72 °C (10 min). 2 mL of PCR products from each well were pooled and purified using the DNA Clean & Concentrator (Zymo) and eluted in water. The resulting libraries were sequenced on an Illumina MiSeq using a MiSeq Reagent Kit v3 (600-cycle) to generate 2× 300 paired-end reads.

### Bioinformatic Analysis for Quantification of Species Abundance

Sequencing data were demultiplexed using Basespace Sequencing Hub's FastQ Generation program. Custom python scripts were used for further data processing (method adapted from Venturelli et al. Mol. Syst. Biol., 2018) 16. Paired end reads were merged using PEAR (v0.9.10) 83 after which reads without forward and reverse annealing regions were filtered out. A reference database of the V3-V5 16S rRNA gene sequences was created using consensus sequences from next-generation sequencing data or Sanger sequencing data of monospecies cultures. Sequences were mapped to the reference database using the mothur (v1.40.5) 84 command classify.seqs (Wang method with a bootstrap cutoff value of 60). Relative abundance for each species within a sample was calculated as the read count mapped to each species divided by the total number of reads for each sample. Absolute abundance of each species was calculated by multiplying the relative abundance by the OD600 measurement for each sample. Samples were excluded from further analysis if >1% of the reads were assigned to a species not expected to be in the community (indicating contamination) or if they had <1000 total reads and OD600 >0.1 (indicating that there were insufficient reads for analysis and this was not due to lack of community growth).

### Consumer Resource Model

Consumer-resource (CR) models are ordinary differential equation models that can be used to model the dynamics microbial species growth on resources. Given a set of nₛ species

$$s = {( {s}_{1} , ... , {s}_{{n}_{s}} )}^{T} ∈ {R}_{+}^{{n}_{s}}$$

and nₘ resources

$$m = {( {m}_{1} , ... , {m}_{{n}_{m}} )}^{T} ∈ {R}_{+}^{{n}_{m}} ,$$

the proposed CR model is the following set of ordinary differential equations

$$\frac{{ds}_{i}}{dt} = {s}_{i} \cdot ( {g}_{i} \cdot {\sum }_{j = 1}^{{n}_{m}} {C}_{ij} {m}_{j} - {d}_{i} ) \qquad ( 1 )$$

$$\frac{{dm}_{j}}{dt} = - {m}_{j} \cdot {\sum }_{i = 1}^{{n}_{s}} {s}_{i} {C}_{ij}$$

where Cᵢⱼ∈R₊ is the consumption rate of species i on resource j, gᵢ∈[0,1] is the species growth efficiency, and dᵢ∈R₊ is the species death rate. The set of model parameters is denoted as θ={Cᵢⱼ, gᵢ, dᵢ: i=1, . . . , nₛ, j=1, . . . , nₘ}. We denote an experimental condition as q, which defines the species and resource initial conditions and measurement time after inoculation. An experimental measurement of species j abundance in experimental condition q is denoted as yⱼ(q) and the corresponding model prediction is denoted as fⱼ(q, θ).

### Defining the Likelihood

We use the CR model to predict the expected value of an outcome from a particular experimental condition and characterize the noise as a Gaussian random variable as

yⱼ(qᵢ)=fⱼ(qᵢ,θ)+εⱼ (2)

where

$${ε}_{j} ∼ \text{?} ( 0 , {σ}_{j}^{2} )$$

is a random variable that models measurement noise. Another way of expressing Eq. 2 is as a likelihood function,

$$p ( {y}_{j} ( {q}_{i} ) \vert θ , {σ}_{j}^{2} ) = \text{?} ( {y}_{j} ( {q}_{i} ) \vert {f}_{j} ( {q}_{i} , θ ) , {σ}_{j}^{2} ) . \qquad ( 3 )$$

An experimental design is a set of experimental conditions, q={q₁, . . . , qₙ}, and data collected from an experimental design is denoted as [symbol] (q)={y(q₁), . . . , y(qₙ)}. Because measurements from each experimental condition are assumed to be drawn independently from Eq. 3, the likelihood of a set of measurements is

$$p ( 𝒟 ( q ) \vert θ , {σ}_{1}^{2} , ... , {σ}_{{n}_{s}}^{2} ) = {\prod }_{i = 1}^{n} {\prod }_{j = 1}^{{n}_{s}} \text{?} ( {y}_{j} ( {q}_{i} ) \vert {f}_{j} ( {q}_{i} , θ ) , {σ}_{j}^{2} ) . \qquad ( 4 )$$

### Variational Inference and Expectation-Maximization for Parameter Inference and Hyper-Parameter Optimization

The goal of variational inference is to optimize a parameter distribution so that it approximates the true parameter posterior distribution, p(θ [symbol] (q)). This is accomplished by optimizing the parameters, denoted as φ, of an approximate probability density function, z(θ|φ)≈p(θ| [symbol] (q)). We will use an independent Gaussian prior distribution for each parameter, p(θₖ|αₖ)= [symbol] (θₖ|0,1/αₖ), where αₖ is the precision (inverse variance) of θₖ.

The set of variables,

$$ξ = \{ {α}_{1} , ... , {α}_{{n}_{θ}} , {σ}_{1}^{2} , ... , {σ}_{{n}_{s}}^{2} \} ,$$

are considered hyper-parameters, because they influence parameter inference but are not explicitly a part of the model. These hyper-parameters control the degree of regularization, which is the amount of penalty placed on parameter values that deviate from the mean of the prior. Because the prior mean is zero in this case, increasing regularization promotes simplicity in the model in the form of zero-valued parameters. The objective function that takes into account model hyper-parameters, ξ, is the log marginal likelihood given by

log p( [symbol] (q)|ξ)=log∫_{θ}p( [symbol] (q),θ|ε)dθ.

For any choice of z(θ|φ), the log marginal likelihood can be decomposed into two functions,

$$log p ( 𝒟 ( q ) \vert ξ ) = {\underbrace{{\int }_{θ} log ( \frac{p ( 𝒟 ( q ) , θ \vert ξ )}{z ( θ \vert ϕ )} ) z ( θ \vert ϕ ) d θ}}_{ℒ ( z ( θ \vert ϕ ) , ξ )} + {\underbrace{{\int }_{θ} - log ( \frac{p ( θ \vert 𝒟 ( q ) , ξ )}{z ( θ \vert ϕ )} ) z ( θ \vert ϕ ) d θ}}_{KL} . \qquad ( 6 )$$

where KL is the Kullback-Leibler divergence between the approximating distribution z(θ|φ) and the true posterior parameter distribution p(θ| [symbol] (q),ξ). Because the KL divergence is strictly non-negative, the function [symbol] (z(θ|φ),ξ) is a lower bound on the log marginal likelihood since [symbol] (z(θ|φ),ξ)≤log p( [symbol] (q)|ξ). This lower bound is referred to as the evidence lower bound (ELBO). Optimizing the ELBO with respect to the parameters of the approximate posterior distribution, φ, is called variational inference. The Expectation-Maximization algorithm iterates between an expectation step, which involves variational inference to maximize the ELBO with respect to φ keeping ξ fixed, followed by a maximization step, in which the ELBO is maximized with respect to ξ keeping φ fixed.

### Expectation Step for Independent Gaussian Parameter Posterior

To enable more scalable Bayesian inference, we can use a simpler distribution to approximate the true parameter posterior where parameters are assumed to be independent,

$$z ( θ \vert ϕ ) = {\prod }_{k = 1}^{{n}_{θ}} \text{?} ( {θ}_{k} \vert {μ}_{k} , {v}_{k}^{2} ) .$$

Evaluating the ELBO in this case gives,

$$ℒ ( z ( θ \vert ϕ ) , ξ ) = {\int }_{θ} log ( p ( \text{?} ( q ) \vert θ , {σ}_{1}^{2} , ... , {σ}_{{n}_{s}}^{2} ) ) {\prod }_{k = 1}^{{n}_{θ}} \text{?} ( {θ}_{k} \vert {μ}_{k} , {v}_{k}^{2} ) d θ + {\int }_{θ} log ( \text{?} ( θ \vert 0 , diag ( 1 / α ) ) ) {\prod }_{k = 1}^{{n}_{θ}} \text{?} ( {θ}_{k} \vert {μ}_{k} , {v}_{k}^{2} ) d θ - {\int }_{θ} log ( \text{?} ( θ \vert μ , \sum ) ) {\prod }_{k = 1}^{{n}_{θ}} \text{?} ( {θ}_{k} \vert {μ}_{k} , {v}_{k}^{2} ) d = {\int }_{θ} {\sum }_{i = 1}^{n} {\sum }_{j = 1}^{{n}_{s}} ( - \frac{{( {y}_{j} ( {q}_{i} ) - {f}_{j} ( {q}_{i} , θ ) )}^{2}}{2 {σ}_{j}^{2}} - log {σ}_{j} ) {\prod }_{k = 1}^{{n}_{θ}} \text{?} ( {θ}_{k} \vert {μ}_{k} , {v}_{k}^{2} ) d + {\sum }_{k = 1}^{{n}_{θ}} ( - \frac{1}{2} {α}_{k} {μ}_{k}^{2} - \frac{1}{2} {α}_{k} {v}_{k}^{2} + \frac{1}{2} log {α}_{k} + log {v}_{k} ) \qquad ( 7 )$$

Linearizing the model with respect to parameters about the point θ=μ gives,

fⱼ(qᵢ,θ)≈fⱼ(qᵢ,μ)+gⱼ(qᵢ)^{T}(θ−μ), (8)

where we define gⱼ(qᵢ)=∇_{θ}fⱼ(qᵢ, θ). Using the linearized model to evaluate the expectation in the ELBO gives,

$${\int }_{θ} {\sum }_{i = 1}^{n} {\sum }_{j = 1}^{{n}_{s}} ( - \frac{{( {y}_{j} ( {q}_{i} ) - {f}_{j} ( {q}_{i} , θ ) )}^{2}}{2 {σ}_{j}^{2}} - log {σ}_{j} ) {\prod }_{k = 1}^{{n}_{θ}} \text{?} ( {θ}_{k} \vert {μ}_{k} , {v}_{k}^{2} ) d θ$$

$$\approx {\sum }_{i = 1}^{n} {\sum }_{j = 1}^{{n}_{s}} ( - \frac{{( {y}_{j} ( {q}_{i} ) - {f}_{j} ( {q}_{i} , μ ) )}^{2} + {\sum }_{k = 1}^{{n}_{θ}} {g}_{jk}^{2} ( {q}_{i} ) {v}_{k}^{2}}{2 {σ}_{j}^{2}} - log {σ}_{j} ) . \qquad ( 9 )$$

Putting all of the terms together gives the approximate expression for the ELBO,

$$ℒ ( \text{?} ( θ \vert μ , \sum ) , ξ ) \approx {\sum }_{i = 1}^{n} {\sum }_{j = 1}^{{n}_{s}} ( - \frac{{( {y}_{j} ( {q}_{i} ) - {f}_{j} ( {q}_{i} , μ ) )}^{2} + {\sum }_{k = 1}^{{n}_{θ}} {g}_{jk}^{2} ( {q}_{i} ) {v}_{k}^{2}}{2 {σ}_{j}^{2}} - log {σ}_{j} ) + {\sum }_{k = 1}^{{n}_{θ}} ( - \frac{1}{2} {α}_{k} {μ}_{k}^{2} - \frac{1}{2} {α}_{k} {v}_{k}^{2} + \frac{1}{2} log {α}_{k} + log {v}_{k} ) \qquad ( 10 )$$

Maximizing this expression for the ELBO with respect to $\mu$ gives the same estimate as before,

$${µ}^{*} = arg \backslash underset µ max {\sum }_{i = 1}^{n} {\sum }_{j = 1}^{{n}_{s}} ( - \frac{{( {y}_{j} ( {q}_{i} ) - {f}_{j} ( {q}_{i} , µ ) )}^{2}}{2 {σ}_{j}^{2}} ) - {\sum }_{k = 1}^{{n}_{θ}} ( \frac{{α}_{k} {µ}_{k}^{2}}{2} ) , \qquad ( 11 )$$

and using the estimate for μ*, maximizing the ELBO with respect to

$${v}_{k}^{2}$$

gives,

$${v}_{k}^{* 2} = {( {α}_{k} + {\sum }_{i = 1}^{n} {\sum }_{j = 1}^{{n}_{s}} \frac{{ℊ}_{jk}^{2} ( {q}_{i} )}{{σ}_{j}^{2}} )}^{- 1} . \qquad ( 12 )$$

Optimization of μ is performed using the Newton-CG method from the Scipy minimize function⁸³.

### Maximization Step for Independent Gaussian Parameter Posterior

Maximizing Eq. 10 evaluated using the optimized variational parameters with respect to each αₖ and each

$${σ}_{j}^{2}$$

now gives

$${α}_{k}^{*} = \frac{1}{{μ}_{k}^{* 2} + {v}_{k}^{* 2}} \qquad ( 13 )$$

$$and$$

$${σ}_{j}^{* 2} = \frac{1}{n} {\sum }_{i = 1}^{n} {( {y}_{j} ( {q}_{i} ) - {f}_{j} ( {q}_{i} , {μ}^{*} ) )}^{2} + {\sum }_{k = 1}^{{n}_{θ}} {ℊ}_{jk}^{2} ( {q}_{1} ) {v}_{k}^{* 2} \qquad ( 14 )$$

### Wald Test for Parameter Significance

For a parameter θᵢ, we use a two-tailed Wald test to test whether the parameter is significantly constrained to be non-zero by the experimental data.

Intuitively, the Wald test compares the parameter mean to its standard deviation in order to evaluate whether the peak of the posterior parameter distribution is significantly higher or lower than zero compared to the width of the distribution. We denote the null hypothesis as, H₀:θᵢ=0. The Wald test of size α=0.05 for parameter i rejects H₀ when |Wᵢ|>z_{α/2}, where

$${W}_{2} = \frac{{μ}_{i}^{*}}{{v}_{i}^{*}} . \qquad ( 15 )$$

The p-value for an observed Wald statistic, Wᵢ, is given by p=2Φ(−|Wᵢ|), which is the probability of the Wald statistic being at least as large as the observed value under the null hypothesis⁸⁴.

### Estimation of Posterior Predictive Distribution

Given a posterior parameter distribution, the posterior predictive distribution is found by marginalizing model predictions with respect to parameters,

$$p ( {y}_{i} ( {q}_{i} ) \vert \text{?} ( q ) , ξ ) = \int \text{?} ( {y}_{j} ( {q}_{i} ) \vert {f}_{j} ( {q}_{i} , θ ) , {σ}_{j}^{2} ) z ( θ \vert {μ}^{*} , {v}^{*} ) d θ . \qquad ( 16 )$$

Linearizing the model prediction with respect to the model parameters around μ* allows for the following approximation of the integral in Eq. 16.

$$p ( {y}_{i} ( {q}_{i} ) \vert \text{?} ( q ) , ξ ) = \int \text{?} ( {y}_{j} ( {q}_{i} ) \vert {f}_{j} ( {q}_{i} , {μ}^{*} ) , {σ}_{j}^{2} + {v}^{* 2} \cdot {{ℊ}_{j} ( {q}_{i} )}^{2} ) . \qquad ( 17 )$$

This approximation is referred to as the linear-Gaussian approximation⁸⁵.

### Bayesian Experimental Design

The goal of Bayesian experimental design is to select an experimental design, denoted as q={q₁, . . . , qₙ} that results in data, [symbol] (q)={y(q₁), . . . , y(qₙ)}, that is expected to minimize the spread of the parameter distribution according to Bayes' formula,

$$p ( θ \vert \text{?} ( Q ) ) = \frac{p ( \text{?} ( q ) \vert θ ) p ( θ )}{p ( \text{?} ( q ) ) .} \qquad ( 18 )$$

The variable qᵢ is introduced to denote a particular experimental condition, which could for example be the specification of species to inoculate and resources to include in the media. It is important to note that the prior p(θ) could be the posterior determined from performing inference on previous data. In order to select for an optimal experimental design, we define a utility function, U(q), as the expected information gain (EIG) that would result from updating the current model with a new dataset [symbol] (q). The EIG is the expected Kullback-Leibler divergence between the parameter posterior and the parameter prior distributions, which has an analytical expression in the case of the linear-Gaussian model

$$\begin{matrix}{U}_{E \vert G} ( q ) = - \int \int rln p ( \text{?} ( q ) ) p ( \text{?} ( q ) \vert θ ) d yp ( θ ) d θ \\ = {\sum }_{k = 1}^{{n}_{θ}} ln ( {v}_{k}^{* - 2} + {\sum }_{i = 1}^{n} {\sum }_{j = 1}^{{n}_{s}} \frac{{{ℊ}_{jk}^{2} ( {q}_{i} )}^{2}}{{σ}_{j}^{2}} )\end{matrix} \qquad ( 19 )$$

where

$${ℊ}_{jk} ( {q}_{i} ) = \frac{\partial {f}_{j} ( {q}_{i} , θ )}{\partial θ}$$

is the gradient of the model prediction of experimental condition qᵢ with respect to model parameters.

Given a set of all possible experimental conditions, denoted as [symbol] , we use a Greedy search algorithm to determine the subset of n experimental conditions that maximize Eq. 19, where the first selected experimental condition is given by,

$${q}_{1} = arg \text{?} {\sum }_{k = 1}^{{n}_{θ}} ln ( {v}_{k}^{* - 2} + {\sum }_{j = 1}^{{n}_{s}} \frac{{{ℊ}_{jk} ( q )}^{2}}{{σ}_{j}^{2}} ) \qquad ( 20 )$$

and subsequent experimental conditions are selected according to

$${q}_{1} = arg \text{?} {\sum }_{k = 1}^{{n}_{θ}} ln ( {v}_{k}^{* - 2} + {\sum }_{i = 1}^{l - 1} {\sum }_{j = 1}^{{n}_{s}} \frac{{{ℊ}_{jk} ( q )}^{2}}{{σ}_{j}^{2}} + {\sum }_{j = 1}^{{n}_{s}} \frac{{{ℊ}_{jk} ( q )}^{2}}{{σ}_{j}^{2}} ) \qquad ( 21 )$$

until l=n, where n is the total number of conditions that are experimentally practical to collect.

### Modified Linear Regression Model

A modified linear regression model was used to assess the effect from inoculum species and resources on C. difficile abundance at 24 hours. This model assumes linear influence of the covariates (inoculum species OD and resource concentration) and interactions among the covariates to the variable of interest (C. difficile OD at 24 hours)^{24,86}. The model formulation is as follows:

$$y = w + {\sum }_{i = 1}^{n} {α}_{i} {X}_{i} + {\sum }_{j = 1}^{P} {β}_{j} {M}_{j} + {\sum }_{k = 1}^{N} {\sum }_{l = k}^{N} {ω}_{il} {X}_{i} {M}_{l} + {\sum }_{k = 1}^{N} {\sum }_{l = k}^{N} {γ}_{kl} {X}_{k} {X}_{l} + {\sum }_{i = 1}^{P} {\sum }_{j = i}^{P} {ϵ}_{ij} {M}_{i} {M}_{j}$$

Where X∈ [symbol] ^{N×S} represents inoculum species OD and M∈ [symbol] ^{P×S} represents inoculum resource concentration. In this model we aim to predict y∈ [symbol] ^{S}, the OD of C. difficile at 24 hours. Each parameter will provide insights into the connection of each covariate towards the growth of C. difficile. Specifically, αᵢ signifies the influence of species i, and βⱼ provides insights into the influence of the nutrient j, ωᵢₗ quantifies the effect of the co-occurrence of organism i and nutrient l, γₖₗ quantifies the effect of the co-occurrence of organisms k and l, and ϵᵢⱼ the effect of the co-occurrence of nutrient i and j. This model was developed using the scikit-learn (sklearn) framework⁵⁷. Model prediction performance was assessed by 10-fold cross validation performed over the whole dataset, splitting the dataset using the ShuffleSplit method of sklearn.

### Light Gradient Boosting Model

The Light Gradient Boosting Model is based on the construction of an ensemble regression trees which will decrease the residual of the predictions^{87,88}. The key feature of LightGBM is that the speed of training is more than 10-fold faster than other competing models such as XGBoost⁸⁷. The fitting for the light gradient boosting model is performed using the LightGBM Python package⁸⁷. Hyperparameter tunning is performed through the Optuna Python package, which is a hyperparameter tuner framework with state-of-the-art sampler and has seamless connection with LightGBM through its python API⁵⁸. Model prediction performance was assessed by 10-fold cross validation performed over the whole dataset, splitting the dataset using the ShuffleSplit method of sklearn.

### SHAP Analysis

For insight into species and resource contribution to C. difficile growth, we adopted a machine learning interpretation method named the SHapley Additive exPlanation (SHAP) method⁴¹. This method gives us the faculty to assign negative and positive values to covariates that in a certain experiment contribute to the movement of the prediction against the mean values, therefore we can infer which microorganisms or nutrients have negative or positive contribution on the C. difficile final OD. Calculations of SHAP values are performed through the SHAP Python package⁴¹.

### UHPLC-MS Measurements

Samples were analyzed using an ultrahigh-pressure liquid chromatography (UHPLC-MS) system consisting of a Thermo Scientific Vanquish UHPLC system coupled to a heated electrospray ionization (HESI; using negative polarity) and hybrid quadrupole high-resolution mass spectrometer (Q Exactive Orbitrap; Thermo Scientific). Settings for the ion source were as follows: auxiliary gas flow rate of 10, sheath gas flow rate of 30, sweep gas flowrate of 1, 2.5-Kv spray voltage, 320 °C capillary temperature, and S-lens radiofrequency (RF) level of 50. Nitrogen was used as nebulizing gas by the ion trap source. Liquid chromatography (LC) separation was achieved using a Waters Acquity UPLC BEH C18 column with 1.7-μm particle size, 2.1 by 100 mm in length. Solvent A was 97% water, 3% methanol with 10 mM TBA adjusted to Ph 8.2 by adding ~560 mL glacial acetic acid/L). Solvent B was 100% methanol. The total run time was 25 min with the following gradient: 0- to 2.5-min at 5% solvent B (initial condition), 2.5- to 17-min gradient to 95% solvent B; hold 2.5 min at 95% solvent B; drop to 5% solvent B by 20-min for re-equilibration to initial condition. The flow rate was 200 μL/min throughout. Other LC parameters were as follows: autosampler temperature, 4 °C; injection volume, 10 μL; column temperature, 30 °C The MS method performed a MS1 full scan (70 to 1,000 m/z) in negative mode with a resolution set to 70,000.

### Determination of Metabolite Concentrations

Metabolite quantitation was achieved using standard concentrations of each metabolite ranging from 0.01 mM to 10 mM to generate six-point standard curves. Samples were diluted to 1/100 or 1/1000 for target metabolite signals to be within the standard curve linear range. Standards were purchased from Sigma-Aldrich and dissolved and stored in HPLC grade water at −20 °C Peaks for the following pairs of metabolites could not be resolved and measurements are reported as combined concentrations: sorbitol/mannitol, galactose/mannose, N-acetyl-glucosamine/N-acetyl-galactosamine, trehalose/cellobiose. Resource utilization is calculated by the following equation:

resource utilization=(Cₜ_{₀}_{,sample}−Cₜ_{₂₄}_{,sample})−(Cₜ_{₀}_{,blank}−Cₜ_{₂₄}_{,blank})

Where C represents measured absolute metabolite concentration in either sample or blank at 0 hour (t0) or 24 hour (t24). The normalized fold change is calculated by the following equation:

$$fold change = \frac{{A}_{{t}_{24} , sample} / {A}_{{t}_{0} , sample}}{{A}_{{t}_{24} , blank} / {A}_{{t}_{0} , blank}}$$

Where A represents area under the curve in either sample or blank at 0 hour (t0) or 24 hour (t24).

### Gnotobiotic Mouse Experiments

All germ-free mouse experiments were performed following protocols approved by the University of Wisconsin-Madison Animal Care and Use Committee. The defined diet used for all experiment was Envigo 2018S, containing 18.4% protein, 6.0% fat, 44.2% carbohydrate, 3.8% crude fiber, 14.7% neutral detergent fiber, and 5.5% ash. All strains were grown at 37 °C anaerobically in ABB media (Oxoid) for 24 or 48 hours. All strains for oral gavage were mixed in equal proportions based on OD600 and transferred on ice prior to oral gavage. We used 8-week old C57BL/6 gnotobiotic female mice (wild-type) fed the defined diets 3 days prior to oral gavage. The mice from the same group (4 or 5 mice) were housed in the same biocontainment cages (Allentown Inc.) for the duration of the experiment. Mice were maintained on autoclaved water. One week after community gavage, 2000 CFU of vegetative C. difficile cells were orally gavaged into all mice in all groups. Mice were weighed and fecal samples were collected every 2-3 days after oral gavage for NGS sequencing and C. difficile CFU counting. Two weeks after C. difficile challenge, mice were euthanized, and the cecal contents were collected for NGS sequencing, C. difficile CFU counting, and toxin ELISA assay.

Genomic DNA Extraction from Fecal and Cecal Samples

The DNA extraction for fecal and cecal samples was performed as described previously with some modifications⁸⁹. Fecal samples (~50 mg) were transferred into solvent-resistant screw-cap tubes (Sarstedt Inc) with 500 mL 0.1 mm zirconia/silica beads (BioSpec Products) and one 3.2 mm stainless steel bead (BioSpec Products). The samples were resuspended in 500 ml of Buffer A (200 mM NaCl (DOT Scientific), 20 mM EDTA (Sigma) and 200 mM Tris·HCl Ph 8.0 (Research Products International)), 210 mL 20% SDS (Alfa Aesar) and 500 ml phenol/chloroform/isoamyl alcohol (Invitrogen). Cells were lysed by mechanical disruption with a bead-beater (BioSpec Products) for 3 min twice to prevent overheating. Next, cells were centrifuged for 5 min at 8,000×g at 4 °C, and the supernatant was transferred to a Eppendof tube. We added 60 mL 3M sodium acetate (Sigma) and 600 mL isopropanol (LabChem) to the supernatant and incubated on ice for 1 hr. Next, samples were centrifuged for 20 min at 18,000×g at 4 °C The harvested DNA pellets were washed once with 500 ml of 100% ethanol (Koptec). The remaining trace ethanol in the sample was removed by air drying. Finally, the DNA pellets were then resuspended into 200 ml of AE buffer (Qiagen) left in 4 °C overnight to facilitate dissolving. The crude DNA extracts were purified by a Zymo DNA Clean & Concentrator™-5 kit (Zymo Research). The following PCR amplification and NGS sequencing procedures are the same as previously described for treatment of in vitro samples.

C. difficile Toxin Measurements Using ELISA

The total toxin concentration (including both TcdA and TcdB) in mice cecal contents were determined by comparison to a standard curve using ELISA (tgcBiomics, Germany). Samples compared for statistical analysis in the same experiment were processed in parallel at the same time using the same batch of ELISA kits to minimize batch-to-batch variations and ensure comparable results.

C. difficile CFU Counting from Fecal and Cecal Samples

C. difficile selective plates were prepared by autoclaving C. difficile agar (Oxoid CM0601) and adding defibrinated horse blood (Lampire 7233401, 70 mL/1 L media), norfloxacin (Santa Cruz 215586, 120 μg/mL), moxalactam (Santa Cruz 250419, 320 μg/mL), and erythromycin (Santa Cruz 204742, 100 μg/mL) after the media was cooled to ~55 °C Immediately following fecal or cecal collection from mice, around 1 ml of fresh fecal samples were taken using an inoculating loop and mixed with PBS. The samples were then serially diluted (1:10 dilution) using PBS. Four dilutions of each sample were spotted on C. difficile selective agar plates, with 2 technical replicates per sample. Plates were incubated at 37 °C for 48 h. At this point, colonies were counted in the dilution spot and contained 5-100 colonies. The CFU/mL for each sample was calculated as the average of the 2 technical replicates times the dilution factor. The lower limit of detection for the assay was 20,000 CFU/MI.

### REFERENCE

1. Cornely, O. A., Miller, M. A., Louie, T. J., Crook, D. W. & Gorbach, S. L. Treatment of First Recurrence of Clostridium difficile Infection: Fidaxomicin Versus Vancomycin. Clinical Infectious Diseases 55, S154-S161 (2012).
2. Song, J. H. & Kim, Y. S. Recurrent Clostridium difficile Infection: Risk Factors, Treatment, and Prevention. Gut Liver 13, 16 (2018).
3. Kazemian, N. et al. The trans-kingdom battle between donor and recipient gut microbiome influences fecal microbiota transplantation outcome. Scientific Reports 2020 10:1 10, 1-10 (2020).
4. Wang, S. et al. Systematic Review: Adverse Events of Fecal Microbiota Transplantation. PloS One 11, e0161174 (2016).
5. DeFilipp, Z. et al. Drug-Resistant E. coli Bacteremia Transmitted by Fecal Microbiota Transplant. New England Journal of Medicine 381, 2043-2050 (2019).
6. Mullard, A. FDA approves second microbiome-based C. difficile therapy. Nat Rev Drug Discov 22, 436 (2023).
7. Louie, T. et al. VE303, a Defined Bacterial Consortium, for Prevention of Recurrent Clostridioides difficile Infection: A Randomized Clinical Trial. JAMA 329, 1356-1366 (2023).
8. Dsouza, M. et al. Colonization of the live biotherapeutic product VE303 and modulation of the microbiota and metabolites in healthy volunteers. Cell Host Microbe 30, 583-598.e8 (2022).
9. Furuichi, M. et al. Commensal consortia decolonize Enterobacteriaceae via ecological control. Nature 2024 633:8031 633, 878-886 (2024).
10. Aguirre, A. M. et al. Bile acid-independent protection against Clostridioides difficile infection. PloS Pathog 17, e1010015 (2021).
11. Jenior, M. L., Leslie, J. L., Young, V. B. & Schloss, P. D. Clostridium difficile Colonizes Alternative Nutrient Niches during Infection across Distinct Murine Gut Microbiomes. mSystems 2, (2017).
12. Marshall, A., McGrath, J. W., Graham, R. & McMullan, G. Food for thought—The link between Clostridioides difficile metabolism and pathogenesis. PloS Pathog 19, e1011034 (2023).
13. Battaglioli, E. J. et al. Clostridioides difficile uses amino acids associated with gut microbial dysbiosis in a subset of patients with diarrhea. Sci Transl Med 10, (2018).
14. Engevik, M. A. et al. Mucin-Degrading Microbes Release Monosaccharides That Chemoattract Clostridioides difficile and Facilitate Colonization of the Human Intestinal Mucus Layer. ACS Infect Dis 7, 1126 (2020).
15. Pruss, K. M. & Sonnenburg, J. L. C. difficile exploits a host metabolite produced during toxin-mediated disease. Nature 2021 593:7858 593, 261-265 (2021).
16. Ferreyra, J. A. et al. Gut Microbiota-Produced Succinate Promotes C. difficile Infection after Antibiotic Treatment or Motility Disturbance. Cell Host Microbe 16, 770-777 (2014).
17. Collins, J. et al. Dietary trehalose enhances virulence of epidemic Clostridium difficile. Nature 2018 553:7688 553, 291-294 (2018).
18. Ghimire, S. et al. Identification of Clostridioides difficile-Inhibiting Gut Commensals Using Culturomics, Phenotyping, and Combinatorial Community Assembly. MSystems 5, (2020).
19. Theriot, C. M. et al. Antibiotic-induced shifts in the mouse gut microbiome and metabolome increase susceptibility to Clostridium difficile infection. Nature Communications 2014 5:1 5, 1-10 (2014).
20. Hasan, M. K., Dhungel, B. A. & Govind, R. Characterization of an operon required for growth on cellobiose in Clostridioides difficile. Microbiology (United Kingdom) 167, 001079 (2021).
21. Pereira, F. C. et al. Rational design of a microbial consortium of mucosal sugar utilizers reduces Clostridioides difficile colonization. Nature Communications 2020 11:1 11, 1-15 (2020).
22. Dong, Q. et al. Protection against Clostridioides difficile disease by a naturally avirulent strain. Cell Host Microbe 0, (2024).
23. Sambol, S. P., Merrigan, M. M., Tang, J. K., Johnson, S. & Gerding, D. N. Colonization for the Prevention of Clostridium difficile Disease in Hamsters. J Infect Dis 186, 1781-1789 (2002).
24. Clark, R. L. et al. Design of synthetic human gut microbiome assembly and butyrate production. Nature Communications 2021 12:1 12, 1-16 (2021).
25. Marcobal, A. et al. A metabolomic view of how the human gut microbiota impacts the host metabolome using humanized and gnotobiotic mice. ISME J 7, 1933-1943 (2013).
26. Smirnov, K. S. et al. Challenges of metabolomics in human gut microbiota research. International Journal of Medical Microbiology 306, 266-279 (2016).
27. Neumann-Schaal, M., Jahn, D. & Schmidt-Hohagen, K. Metabolism the difficile way: The key to the success of the pathogen Clostridioides difficile. Front Microbiol 10, 219 (2019).
28. Jenior, M. L., Leslie, J. L., Young, V. B. & Schloss, P. D. Clostridium difficile Colonizes Alternative Nutrient Niches during Infection across Distinct Murine Gut Microbiomes. mSystems 2, (2017).
29. Venturelli, O. S. et al. Deciphering microbial interactions in synthetic human gut microbiome communities. Mol Syst Biol 14, (2018).
30. MacArthur, R. Species packing and competitive equilibrium for many species. Theor Popul Biol 1, 1-11 (1970).
31. Lee, H., Bloxham, B. & Gore, J. Resource competition can explain simplicity in microbial community assembly. Proc Natl Acad Sci USA 120, (2023).
32. Ho, P. Y., Nguyen, T. H., Sanchez, J. M., DeFelice, B. C. & Huang, K. C. Resource competition predicts assembly of gut bacterial communities in vitro. Nature Microbiology 2024 9:4 9, 1036-1048 (2024).
33. Thompson, J. C., Zavala, V. M. & Venturelli, O. S. Integrating a tailored recurrent neural network with Bayesian experimental design to optimize microbial community functions. PloS Comput Biol 19, e1011436 (2023).
34. Sulaiman, J. E. et al. Elucidating human gut microbiota interactions that robustly inhibit diverse Clostridioides difficile strains across different nutrient landscapes. Nature Communications 2024 15:1 15, 1-20 (2024).
35. Procopio, A. et al. Combined mechanistic modeling and machine-learning approaches in systems biology-A systematic literature review. Comput Methods Programs Biomed 240, 107681 (2023).
36. Cai, W. et al. Incorporating microbial community data with machine learning techniques to predict feed substrates in microbial fuel cells. Biosens Bioelectron 133, 64-71 (2019).
37. Thompson, J., Johansen, R., Dunbar, J. & Munsky, B. Machine learning to predict microbial community functions: An analysis of dissolved organic carbon from litter decomposition. PloS One 14, e0215502 (2019).
38. Kim, K. et al. Decision-Tree-based data mining and rule induction for predicting and mapping soil bacterial diversity. Environ Monit Assess 178, 595-610 (2011).
39. Yu, S. II et al. Simulation models of microbial community, Ph, and volatile fatty acids of anaerobic digestion developed by machine learning. Journal of Water Process Engineering 60, 105225 (2024).
40. McElfresh, D. et al. When Do Neural Nets Outperform Boosted Trees on Tabular Data? Adv Neural Inf Process Syst 36, (2023).
41. Lundberg, S. M., Allen, P. G. & Lee, S.-I. A Unified Approach to Interpreting Model Predictions. Doi: 10.5555/3295222.3295230.
42. Lundberg, S. M. et al. Explainable AI for Trees: From Local Explanations to Global Understanding. (2019).
43. Molnar, C. et al. General Pitfalls of Model-Agnostic Interpretation Methods for Machine Learning Models. Lecture Notes in Computer Science (including subseries Lecture Notes in Artificial Intelligence and Lecture Notes in Bioinformatics) 13200 LNAI, 39-68 (2022).
44. Karp, P. D. et al. The BioCyc collection of microbial genomes and metabolic pathways. Brief Bioinform 20, 1085-1093 (2019).
45. Wu, S. et al. Gmrepo: a database of curated and consistently annotated human gut metagenomes. Nucleic Acids Res 48, D545-D553 (2020).
46. Dai, D. et al. Gmrepo v2: a curated human gut microbiome database with special focus on disease markers and cross-dataset comparison. Nucleic Acids Res 50, D777-D784 (2022).
47. Buffie, C. G. et al. Precision microbiome reconstitution restores bile acid mediated resistance to Clostridium difficile. Nature 2014 517:7533 517, 205-208 (2014).
48. Hromada, S. et al. Negative interactions determine Clostridioides difficile growth in synthetic human gut communities. Mol Syst Biol 17, e10355 (2021).
49. Spinler, J. K. et al. Nextgeneration probiotics targeting Clostridium difficile through precursor-directed antimicrobial biosynthesis. Infect Immun 85, (2017).
50. Hanchi, H. et al. Inhibition of MRSA and of Clostridium Difficile by Durancin 61A: Synergy with Bacteriocins and Antibiotics. Future Microbiol 12, 205-212 (2017).
51. Battaglioli, E. J. et al. Clostridioides difficile uses amino acids associated with gut microbial dysbiosis in a subset of patients with diarrhea. Sci Transl Med 10, 7019 (2018).
52. Salyers, A. A., Vercellotti, J. R., West, S. E. H. & Wilkins, T. D. Fermentation of mucin and plant polysaccharides by strains of Bacteroides from the human colon. Appl Environ Microbiol 33, 319-322 (1977).
53. Roberton, A. M. & Stanley, R. A. In vitro utilization of mucin by Bacteroides fragilis. Appl Environ Microbiol 43, 325-330 (1982).
54. Tsai, H. H., Hart, C. A. & Rhodes, J. M. Production of mucin degrading sulphatase and glycosidases by Bacteroides thetaiotaomicron. Lett Appl Microbiol 13, 97-101 (1991).
55. Altman, D. G. & Bland, J. M. Standard deviations and standard errors. BMJ 331, 903 (2005).
56. Liu, Y. et al. The arginine dihydrolase pathway shapes human gut microbial community assembly, functions, and mammalian host colonization. BioRxiv 2023.01.10.523442 (2023) doi:10.1101/2023.01.10.523442.
57. Pedregosa FABIANPEDREGOSA, F. et al. Scikit-learn: Machine Learning in Python Gaël Varoquaux Bertrand Thirion Vincent Dubourg Alexandre Passos PEDREGOSA, VAROQUAUX, GRAMFORT ET AL. Matthieu Perrot. Journal of Machine Learning Research 12, 2825-2830 (2011).
58. Akiba, T., Sano, S., Yanase, T., Ohta, T. & Koyama, M. Optuna: A Next-generation Hyperparameter Optimization Framework. Proceedings of the ACM SIGKDD International Conference on Knowledge Discovery and Data Mining 2623-2631 (2019) doi: 10.1145/3292500.3330701.
59. Britz, M. L. & Wilkinson, R. G. Leucine dissimilation to isovaleric and isocaproic acids by cell suspensions of amino acid fermenting anaerobes: the Stickland reaction revisited. Can J Microbiol 28, 291-300 (1982).
60. Jackson, S., Calos, M., Myers, A. & Self, W. T. Analysis of proline reduction in the nosocomial pathogen Clostridium difficile. J Bacteriol 188, 8487-8495 (2006).
61. Karasawa, T., Ikoma, S., Yamakawa, K. & Nakamura, S. A defined growth medium for Clostridium difficile. Microbiology (N Y) 141, 371-375 (1995).
62. Shrestha, R., Lockless, S. W. & Sorg, J. A. A Clostridium difficile alanine racemase affects spore germination and accommodates serine as a substrate. Journal of Biological Chemistry 292, 10735-10742 (2017).
63. Girinathan, B. P., Braun, S., Sirigireddy, A. R., Lopez, J. E. & Govind, R. Importance of Glutamate Dehydrogenase (GDH) in Clostridium difficile Colonization In Vivo. PloS One 11, e0160107 (2016).
64. Fachi, J. L. et al. Butyrate Protects Mice from Clostridium difficile-Induced Colitis through an HIF-1-Dependent Mechanism. CellReports 27, 750-761.e7 (2019).
65. Chen, X. et al. A Mouse Model of Clostridium difficile-Associated Disease. Gastroenterology 135, 1984-1992 (2008).
66. Kokkotou, E. et al. Comparative Efficacies of Rifaximin and Vancomycin for Treatment of Clostridium difficile-Associated Diarrhea and Prevention of Disease Recurrence in Hamsters. Antimicrob Agents Chemother 52, 1121 (2008).
67. Jenior, M. L. et al. Systems-ecology designed bacterial consortium protects from severe Clostridioides difficile infection. BioRxiv 2023.08.08.552483 (2023) doi: 10.1101/2023.08.08.552483.
68. Freschlin, C. R., Fahlberg, S. A., Heinzelman, P. & Romero, P. A. Neural network extrapolation to distant regions of the protein fitness landscape. Nature Communications 2024 15:1 15, 1-13 (2024).
69. Leslie, J. L., Vendrov, K. C., Jenior, M. L. & Young, V. B. The Gut Microbiota Is Associated with Clearance of Clostridium difficile Infection Independent of Adaptive Immunity. MSphere 4, (2019).
70. Zampieri, M., Sekar, K., Zamboni, N. & Sauer, U. Frontiers of high-throughput metabolomics. Curr Opin Chem Biol 36, 15-23 (2017).
71. Vignoli, A. et al. High-Throughput Metabolomics by 1D NMR. Angewandte Chemie International Edition 58, 968-994 (2019).
72. Connors, J., Dawe, N. & Van Limbergen, J. The Role of Succinate in the Regulation of Intestinal Inflammation. Nutrients 2019, Vol. 11, Page 25 11, 25 (2018).
73. Wei, Y. han, Ma, X., Zhao, J. C., Wang, X. Q. & Gao, C. Q. Succinate metabolism and its regulation of host-microbe interactions. Gut Microbes 15, (2023).
74. Hoel, D. G. & Walburg, H. E. Statistical Analysis of Survival Experiments. JNCI: Journal of the National Cancer Institute 49, 361-372 (1972).
75. Festing, M. F. W. & Altman, D. G. Guidelines for the Design and Statistical Analysis of Experiments Using Laboratory Animals. ILAR J 43, 244-258 (2002).
76. Nguyen, V. K. et al. Enzyme immunoassay (ELISA) for detection of Clostridium difficile toxin B in specimens of faeces. J Med Microbiol 31, 251-257 (1990).
77. Lyerly, D. M., Sullivan, N. M. & Wilkins, T. D. Enzyme-linked immunosorbent assay for Clostridium difficile toxin A. J Clin Microbiol 17, 72-78 (1983).
78. Roshan, N., Riley, T. V., Knight, D. R. & Hammer, K. A. Effect of natural products on the production and activity of Clostridium difficile toxins in vitro. Scientific Reports 2018 8:1 8, 1-9 (2018).
79. Mahony, D. E., Gilliatt, E., Dawson, S., Stockdale, E. & Lee, S. H. S. Vero cell assay for rapid detection of Clostridium perfringens enterotoxin. Appl Environ Microbiol 55, 2141-2143 (1989).
80. Icho, S. et al. Intestinal bile acids provide a surmountable barrier against C. difficile TcdB-induced disease pathogenesis. Proc Natl Acad Sci USA 120, e2301252120 (2023).
81. Schora, D. M., Peterson, L. R. & Usacheva, E. A. Immunological Stability of Clostridium difficile Toxins in Clinical Specimens. Infect Control Hosp Epidemiol 39, 434-438 (2018).
82. Paredes-Sabja, D., Shen, A. & Sorg, J. A. Clostridium difficile spore biology: Sporulation, germination, and spore structural proteins. Trends Microbiol 22, 406-416 (2014).
83. Virtanen, P. et al. SciPy 1.0: fundamental algorithms for scientific computing in Python. Nature Methods 2020 17:3 17, 261-272 (2020).
84. Wasserman, L. et al. A Concise Course in Statistical Inference.
85. Bishop, C. M. Prml. (2006).
86. Roustaei, N. Application and interpretation of linear-regression analysis. Medical Hypothesis, Discovery and Innovation in Ophthalmology 13, 151 (2024).
87. Ke, G. et al. LightGBM: A Highly Efficient Gradient Boosting Decision Tree. Adv Neural Inf Process Syst 30, (2017).
88. Chen, T. & Guestrin, C. XGBoost: A Scalable Tree Boosting System. Proceedings of the ACM SIGKDD International Conference on Knowledge Discovery and Data Mining 13-17 Aug. 2016, 785-794 (2016).
89. Goodman, A. L. et al. Extensive personal human gut microbiota culture collections characterized and manipulated in gnotobiotic mice. Proc Natl Acad Sci USA 108, 6252-6257 (2011).

1. A composition comprising a bacterial community, wherein the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

2. The composition of claim 1, wherein the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

3. The composition of claim 1, wherein the bacterial community comprises a purified bacterial strain of each of any two or more, any three or more, or any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

4. The composition of claim 1, wherein the bacterial community comprises a purified bacterial strain of each of any four or more of Escherichia coli (EC), Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

5. The composition of claim 1, wherein the bacterial community comprises a purified bacterial strain of each of:
Escherichia coli (EC); and
any two or more or any three or more of Bacteroides uniformis (BU), Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

6. The composition of claim 1, the bacterial community comprises a purified bacterial strain of each of:
Escherichia coli (EC);
Bacteroides uniformis (BU); and
any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), Roseburia intestinalis (RI), Dorea formicigenerans (DF), Blautia hydrogenotrophica (BH), Clostridium scindens (CS), Dorea longicatena (DL), and Desulfovibrio piger (DP).

7. The composition of claim 1, wherein the bacterial community comprises a purified bacterial strain of each of:
Escherichia coli (EC);
Bacteroides uniformis (BU); and
any one or more or any two or more of Bacteroides thetaiotaomicron (BT), Bacteroides ovatus (BO), Bacteroides caccae (BC), Bacteroides fragilis (BF), Collinsella aerofaciens (CA), Coprococcus comes (CC), Faecalibacterium prausnitzii (FP), Segatella copri (PC), and Roseburia intestinalis (RI).

8. The composition of claim 1, wherein the bacterial community comprises a purified bacterial strain of each of:
Escherichia coli (EC); and
any one or more, any two or more, or each of Bacteroides uniformis (BU), Bacteroides fragilis (BF), and Roseburia intestinalis (RI).

9. The composition of claim 1, wherein the bacterial community consists of individual bacterial cells of the purified bacterial strains and a number of the individual bacterial cells in the bacterial community is at least 50% of a total number of individual bacterial cells in the composition.

10. The composition of claim 1, wherein no more than 5% of a total number of individual bacterial cells in the composition are bacterial strains of Acidaminococcus fermentans, Acidaminococcus intestine, Anaerostipes hadrus, Anaerotruncus colihominis DSM 17241, Anaerotruncus colihominis, Bifidobacterium bifidum, Blautia coccoides, Blautia faecis, Blautia hansenii, Blautia luti, Blautia producta ATCC 27340, Blautia producta, Blautia wexlerae, Clostridium bacterium UC5.1-1D4, Clostridium bolteae 90A9, Clostridium bolteae, Clostridium disporicum, Clostridium hathewayi, Clostridium hiranonis, Clostridium innocuum, Clostridium orbiscindens 1_3_50AFAA, Clostridium symbiosum WAL-14163, Clostridium symbiosum, Dracourtella massiliensis GDI, Dracourtella massiliensis, Eisenbergiella tayi, Erysipelatoclostridium ramosum, Erysipelotrichaceae bacterium_21_3, Eubacterium contortum, Eubacterium fissicatena, Eubacterium rectale, Flavinofractor plautii, Fusicatenibacter saccharivorans, Lachnospiraceae bacterium 7-1 58FAA, Lactobacillus mucosae, Marvinbryantaformexigens, Megasphaera elsdenii, Pseudoflavinofractor capillosus, Roseburia faecis, Ruminococcus albus, Ruminococcus champanellensis, Ruminococcus faecis, Ruminococcus obeum, Ruminococcus torques, Sellimonas intestinalis, Subdoligranulum, and Turicibacter sanguinis.

11. The composition of claim 1, wherein no more than 5% of a total number of individual bacterial cells in the composition are bacterial strains of Parabacteroides johnsonii (PJ), Phocaeicola vulgatus (PV), Bacteroides cellulosilyticus (BY), Enterocloster asparagiformis (CG), Anaerobutyricum hallii (EH), Anaerostipes caccae (AC), Agathobacter rectalis (ER), Peptacetobacter hiranonis (CH), Eggerthella lenta (EL), Bifidobacterium pseudocatenulatum (BP), Bifidobacterium longum (BL), and Bifidobacterium adolescentis (BA).

12. The composition of claim 1, wherein no more than 5% of a total number of individual bacterial cells in the composition are bacterial strains of Bacteroides thetaiotaomicron (BT), Bacteroides ovatus (BO), Bacteroides caccae (BC), Collinsella aerofaciens (CA), Coprococcus comes (CC), Faecalibacterium prausnitzii (FP), Segatella copri (PC), Dorea formicigenerans (DF), Blautia hydrogenotrophica (BH), Clostridium scindens (CS), Dorea longicatena (DL), and Desulfovibrio piger (DP).

13. The composition of claim 1, wherein no more than 5% of a total number of individual bacterial cells in the composition are bacterial strains of Clostridium hiranonis, Bacteroides thetaiotaomicron, Bacteroides uniformis, Phocaeicola vulgatus, Clostridium scindens, Collinsella aerofaciens, and Desulfovibrio piger.

14-18. (canceled)

19. The composition of claim 1, wherein the bacterial community comprises bacterial strains purified from different sources.

20. The composition of claim 1, wherein the bacterial community inhibits any one or more of C. difficile toxin production, C. difficile replication, and C. difficile survival.

21. (canceled)

22. The composition of claim 1, wherein the composition is in the form of a pharmaceutical composition comprising the bacterial community in combination with a pharmaceutically acceptable excipient.

23. The composition of claim 22, wherein the composition is formulated for oral or rectal delivery.

24. The composition of claim 22, wherein the composition is formulated for delivery to the intestine or the colon.

25. The composition of claim 1, wherein the composition is in the form of a food product comprising the bacterial community and a nutrient.

26. A method of treating a C. difficile infection in a subject, comprising administering to the subject a therapeutically effective amount of the composition of claim 1 to treat the C. difficile infection.

27-30. (canceled)
